(19) United States
(12) Patent Application Publication
(10) Pub. No.: US 2003/0203868 A1
(43) Pub. Date: Oct. 30, 2003

(54) Inhibition of pathogen replication by RNA interference

(76) Inventors: Frederic D. Bushman, Encinitas, CA (US); Wen-Yuan Hu, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/361,161

(22) Filed: Feb. 6, 2003

Related U.S. Application Data
(60) Provisional application No. 60/354,684, filed on Feb. 6, 2002.

(51) Int. Cl. A61K 48/00; A61K 39/12; A61K 39/21

(52) U.S. Cl. 514/44; 424/208.1; 424/204.1

(57) ABSTRACT

A method and composition for the treatment of pathogenic diseases was developed using the mechanism of RNA interference. The method uses double-stranded RNA to activate the RNA interference pathways within mammalian or pathogen cells. The method can be used to treat any diseases which are caused by or associated with pathogens. A method for identifying double-stranded RNAs useful for the treatment of pathogenic diseases is also presented, as well as model systems which allow this identification. Also described are methods in which siRNAs are used for the inhibition of HIV replication in human cells, as well as the inhibition of RSV pathogenesis in chick embryos.

This application claims the benefit of priority to provisional application serial No. 60/354,684, filed Feb. 6, 2002, the entire disclosure of which is incorporated herein by reference.

Certain aspects of the invention disclosed herein were made with United States government support under National Institutes of Health grants GM 56553, and AI 34786. The United States government may have certain rights in these aspects of the invention.

### BACKGROUND OF THE INVENTION

The present invention relates generally to a method of inhibiting pathogen replication and, more specifically, to the treatment of pathogen-induced diseases and conditions by RNA interference using complementary double-stranded RNAs.

According to the World Health Organization, infectious diseases account for more than 13 million deaths every year. Although the great majority of these deaths occur in developing countries, infectious diseases do not recognize international boundaries. New diseases have emerged, others once viewed as declining in significance have resurged in importance, and many diseases have developed substantial resistance to known antimicrobial drugs. This picture is complicated by the potential deployment of infectious disease pathogens as weapons of war or instruments of terror.

Pathogenesis refers both to the mechanism of infection and to the mechanism by which disease develops. Pathogens are organisms that cause disease and include, viruses, bacteria, fungi and parasites. Pathogenic mechanisms of viral disease include implantation of the virus at a body site (the portal of entry), replication at that site, and then spread to and multiplication within sites (target organs) where disease or shedding of virus into the environment occurs. Antiviral drugs work by interfering with the viral enzymes, but currently are effective only against a very small number of viral diseases.

Bacteria can cause a multitude of different infections, ranging in severity from undetectable to fulminating. The capacity of a bacterium to cause disease reflects its relative pathogenicity. The body reacts to pathogenic bacteria by local inflammation and by sending in cells from the immune system to attack and destroy the bacteria. Serious infections can be treated with antibiotics, which work by disrupting the bacterium's metabolic processes. While many bacterial infections can be treated successfully with appropriate antibiotics, an increasing number of antibiotic-resistant strains is beginning to emerge. Thus, new treatment modalities are needed for both viral and bacterial pathogens.

RNA interference (RNAi) methodologies hold tremendous promise with regard to selective inhibition of gene expression in vertebrates. RNAi is an innate cellular process that is activated when a double-stranded RNA (dsRNA) molecule of greater than 19 duplex nucleotides enters the cell, causing the degradation of not only the invading dsRNA molecule itself, but also single-stranded RNAs of identical sequences, including endogenous mRNAs. As such, RNAi is a powerful tool in the development of highly specific RNA-based gene-silencing therapeutics.

A need exists for creating flexible molecular tools that can exploit newly sequenced pathogenic genomes and combat pathogenic disease caused by these infectious agents. The present invention satisfies this need and provides related advantages as well.

### SUMMARY OF THE INVENTION

A method and composition for the treatment of pathogenic diseases was developed that exploits the innate cellular pathway of RNA interference. The method uses double-stranded RNA to activate the RNA interference pathways within host cells, for example, vertebrate cells, or within the pathogenic cells themselves. The method can be used to treat any diseases which are caused by, or associated with, pathogens. A method for identifying new double-stranded RNAs useful for the treatment of pathogenic diseases is also presented, as well as a model system that allows for this identification.

In one embodiment, the invention provides a method for inhibiting the growth of a pathogen by contacting the pathogen with a double-stranded RNA (dsRNA) molecule that corresponds to a target gene essential to growth of the pathogen and incubating the dsRNA molecule and the pathogen under conditions that result RNA interference, thereby inhibiting the growth of the pathogen.

In another embodiment, the invention is directed to a method for identifying a gene sequence that is a target for RNA interference aimed at inhibiting the growth of a pathogen by selecting a candidate target gene sequence; contacting a host cell containing a pathogen with a dsRNA that corresponds to the target gene sequence; and identifying whether the dsRNA inhibits the growth of the pathogen.

In a further embodiment, the invention provides a method for inhibiting the growth of a pathogen in an organism by administering to the organism a double-stranded RNA (dsRNA) molecule that corresponds to a target gene, wherein the target gene is essential to growth of the pathogen.

The invention also provides a method of making a transgenic animal capable of expressing a dsRNA that corresponds to a target gene in a pathogen by the steps of identifying a target gene in the pathogen; preparing a nucleic acid sequence having a region that corresponds to a portion of the target gene, wherein the nucleic acid is able to form a dsRNA once expressed in the animal; contacting a recipient animal with the nucleic acid; producing one or more offspring of the recipient animal; and testing the offspring for expression of the dsRNA. In a related embodiment, the invention provides transgenic organisms prepared by the methods described herein.

The invention also provides a number of compositions encompassing dsRNA molecules that correspond to a portion of the HIV genome, preferably, gag, pol or env.

### BRIEF DESCRIPTION OF THE DRAWINGS

FIGS. 1A-1D show bar graphs illustrating RNAi in human cell lines. FIG. 1A shows RNAi suppression of luciferase activity assay by cotransfection. FIG. 1B shows RNAi suppression of green fluorescence protein activity assayed by cotransfection. In FIGS. 1A and 1B, the siRNAs indicated beneath each bar graph were cotransfected with a DNA encoding luc (A) or gfp (B). FIG. 1C shows RNAi suppression of luciferase activity in cells stably transduced with a luc-expression vector. FIG. 1D shows RNAi suppression of green fluorescent protein activity in cells stably transduced with a GFP-expression vector. In FIGS. 1C and 1D cells were first stably transduced with a lentiviral vector transducing the indicated marker gene, and siRNAs were subsequently introduced by transfection.

FIG. 2 shows a line graph illustrating the persistence of RNAi in HOS cells (open symbols) or 293T cells (filled symbols) stably transduced with the GFP gene were treated with siRNA against gfp or luc, and samples were assayed for GFP activity as a function of time after treatment.

FIG. 3 is a schematic diagram of an assay for siRNA inhibition of HIV-1 replication.

FIG. 4 is a bar graph that shows inhibition of HIV replication by RNAi. Inhibition was measured using the indicated siRNAs.

FIG. 5 is a bar graph showing the inhibition of HIV replication assayed with the LTR-luc reporter. All values were normalized to the no siRNA sample. “ss” indicates sense strand only of the siRNA, “as” indicates antisense strand only, and “ds” indicates the complete double-stranded siRNA.

FIG. 6 is a bar graph showing that RNAi blocks retroviral gene expression late during infection.

FIG. 7 is a bar graph showing the output of HIV-1 from infected HOS.T4.CXCR4 cells scored by quantitating HIV-1 p24 (capsid antigen) in the culture supernatant. “Control” indicates no virus in the initial infection.

FIG. 8 is a bar graph showing that RNAi does not act early against incoming viral RNA genomes.

FIG. 9 is a bar graph showing RNA interference in chick embryos.

FIG. 10 is a bar graph showing inhibition of RSV replication by RNA interference.

FIG. 11A is a bar graph showing the quantitation of cells stained with the mpm3 marker, which detects tyrosine phosphorylation characteristic of mitosis, and demonstrates the inhibition of RSV pathogenesis by RNAi by comparing staining on the electroporated (+) and control (−) sides of the neural tube.

FIG. 11B is a bar graph demonstrating the inhibition of RSV pathogenesis by RNAi by showing the quantitation of cells stained with the kip1 marker, which detects the kip1 protein that inhibits cell cycle progression and marks postmitotic cells.

FIG. 12 is a bar graph showing that RNAi does not inhibit the accumulation of viral cDNA early after infection.

### DETAILED DESCRIPTION OF THE INVENTION

This invention is directed to a method and composition for inhibiting the replication of a pathogen in vertebrate cells. In particular embodiments, the invention is directed to inhibiting a viral infection in vertebrates. The methods encompassed by the present invention utilize the innate process of RNA interference (RNAi) to selectively block pathogen replication.

In one embodiment, the invention provides a method for inhibiting the growth of a pathogen by contacting the pathogen with a double-stranded RNA (dsRNA) molecule that corresponds to a target gene essential to growth of the pathogen and incubating the dsRNA molecule and the pathogen under conditions that result in RNA interference, thereby inhibiting the growth of the pathogen.

In particular embodiments of the invention, the pathogen is contained in a host cell when it is contacted with the dsRNA molecule. This is particularly applicable to pathogens, for example, viruses or transposons, that do not have the machinery necessary for RNA interference to occur. In other embodiments, where the pathogen possesses the necessary machinery for RNA interference to occur, the pathogen is contacted directly with the dsRNA. The contacting of a pathogen contained in cell can occur in vitro as well as in vivo.

In certain embodiments, the pathogen that is inhibited via the methods of the invention is a retrovirus. This embodiment is based, in part, on the surprising discovery that RNAi can inhibit retroviral replication most efficiently late during infection rather than immediately upon viral entry into the cell.

As used herein, the term “RNA interference” or “RNAi” refers to the sequence specific silencing of a target gene that is induced by the presence of a dsRNA molecule which corresponds to a portion of the target gene nucleic acid sequence and induces the degradation of mRNA transcribed from the target gene. RNAi can be induced by incubating the dsRNA molecule and pathogen under conditions whereby the target genes' mRNA is digested or modified by other means, thereby preventing translation of the gene product. RNAi is a natural phenomenon believed to occur in the nematode Caenorhabditis elegans, in the fruit fly Drosophila melanogaster, and in some plant species. It most likely serves to protect organisms from viruses, and suppress the activity of transposons, segments of DNA that can move from one location to another, sometimes causing production of an abnormal gene product. An intermediate in the RNAi process, siRNA can be effective in degrading mRNA and, therefore, carries the potential to specifically degrade endogenous mRNA that corresponds to a target gene and thereby inhibit its expression. The strand of the siRNA that is identical in sequence to a region on a target gene transcript is often referred to as the sense strand, while the other strand, which is complementary, is frequently termed the antisense strand.

In RNA interference as it occurs naturally, during the initiation step, input dsRNA is digested into 21-23 nucleotide small interfering RNAs (siRNAs), which have also been called “guide RNAs” as described in Hammond et al. Nature Rev Gen 2: 110-119 (2001); Sharp, Genes Dev 15: 485-490 (2001); and Hutvagner and Zamore, Curr Opin Genetics & Development 12:225-232( 2002), which are incorporated herein by reference in their entirety. The siRNAs are produced when an enzyme belonging to the RNase III family of dsRNA-specific ribonucleases progressively cleaves dsRNA, which can be introduced directly or via a transgene or vector. Successive cleavage events degrade the RNA to 19-21 base pair duplexes (siRNAs), each with 2-nucleotide 3′ overhangs as described by Hutvagner and Zamore, Curr. Opin. Genetics & Development 12:225-232 (2002); Bernstein et al., Nature 409:363-366 (2001), which are incorporated herein by reference in their entirety. In the effector step, the siRNA duplexes bind to a nuclease complex to form what is known as the RNA-induced silencing complex, or RISC. The active RISC then targets the homologous transcript by base pairing interactions and cleaves the mRNA approximately 12 nucleotides from the 3′ terminus of the siRNA (Nykanen et al., Cell 107:309-321 (2001), which is incorporated herein by reference in its entirety).

In most host cells longer dsRNA provokes a non-specific cytotoxic response. In contrast, the introduction of shorter dsRNAs, in particular siRNAs, appears to suppress gene expression without producing a non-specific cytotoxic response because the small size of the siRNAs, as compared to larger dsDNA, prevents activation of the dsRNA-inducible interferon system in mammalian cells and avoids the non-specific phenotypes that can be observed by introducing larger dsRNA.

As described herein, double-stranded RNA is used to activate the RNA interference pathway within vertebrate cells, or the cells of a pathogen. The method can be used to treat any disease or condition that is caused by or associated with a pathogen. A method for identifying double-stranded RNAs useful for the treatment of pathogenic diseases is also presented, as well as a model system which allows this identification.

Thus, one embodiment of the invention relates to the use of double-stranded siRNAs as treatments of conditions or diseases caused by pathogens. In another embodiment, the invention is directed to a method for identifying a gene sequence that is a target for RNA interference aimed at inhibiting the growth of a pathogen by selecting a candidate target gene sequence; contacting a host cell containing a pathogen with a dsRNA that corresponds to the target gene sequence; and identifying whether the dsRNA inhibits the growth of the pathogen.

The methods of the invention can be used to ameliorate a sign and/or symptom associated with a disease or condition caused by a pathogen. As used herein, the terms “disease” and “condition” refer to an interruption, cessation or deviation from the normal structure or function of any part, organ, or system of the body. One skilled in the art can readily recognize signs or symptoms associated with a disease or condition and can readily recognize the amelioration of an associated sign and/or symptom. The methods of the invention can be applied to the treatment of a variety of pathogen-induced diseases or conditions as described in further detail below. Pathogenic infection refers to the colonization and/or invasion and multiplication of pathogenic microrganisms in the host with or without the manifestation of disease.

As used herein, “long” dsRNAs refer to those which are longer than typical siRNAs, longer than about 23 nucleotides and are processed to be used as primers. Similarly, “short” double-stranded RNAs are siRNAs which can be used as primers for RNAi. Methods for making the “long” or “short” dsRNAs are discussed below, but can be any methods known to one skilled in the art. Therefore, the term “dsRNA” encompasses molecules of the size referred to in the art as siRNAs as well as larger RNA duplexes, as long as functionality with regard to pathogen inhibition via target gene silencing is preserved.

As used herein, a double-stranded RNA corresponding to a target gene refers to a double-stranded RNA copy that, except for possessing Uracil instead of Thymine, has substantially the same nucleic acid sequence as a portion of the DNA duplex that encodes a target gene on its coding strand, which is also referred to as non-template strand, plus strand, or sense strand. Thus, a double-stranded RNA corresponding to a target gene transcript has one strand that has substantially the sequence that would result during mRNA synthesis from the template or anti-sense strand, which corresponds to a portion of the target gene, and its complementary sequence.

A dsRNA corresponding to a target gene can have, for example, between 50 and 100 contiguous base pairs, between 25 and 50 contiguous base pairs, between 14 and 26 contiguous base pairs that correspond to the target gene, between 15 and 25, between 16 and 24, between 17 and 23, between 18 and 22, between 19 and 21 contiguous base pairs, up to the full length of the corresponding DNA duplex, as long as the dsRNA is capable of specific target gene inhibition. In this regard, the dsRNA corresponding to the target gene can be of any length as long as dsRNA-dependent protein kinase (PKR) is not induced upon formation of the dsRNA. A major component of the mammalian non-specific response to dsRNA is mediated by the dsRNA-dependent protein kinase, PKR, which phosphorylates and inactivates the translation factor eIF2a, leading to a generalized suppression of protein synthesis and cell death via both nonapoptotic and apoptotic pathway. PKR can be one of several kinases in mammalian cells that can mediate this response.

As used herein, the term “pathogen” refers to any infectious replicating agent causing disease in an organism. In one embodiment, the pathogens to be targeted are viruses, including RNA viruses such as flaviviruses, picornaviruses, rhabdoviruses, filoviruses, retroviruses, including lentiviruses, or DNA viruses such as adenoviruses, poxviruses, herpes viruses, cytomegaloviruses, hepadnaviruses or others. Additional pathogens include bacteria, fungi, helminths, schistosomes and trypanosomes. Other kinds of pathogens can include mammalian transposable elements.

Other embodiments of the invention include methods of treating subjects infected by a pathogen by administering to the subjects a therapeutically effective amount of an siRNA. In particular embodiments, the invention provides a method for inhibiting the growth of a pathogen in an organism by administering to the organism a double-stranded RNA (dsRNA) molecule that corresponds to a target gene, wherein the target gene is essential to the growth of the pathogen. Also provided is a method of treating a pathogenic condition in an animal by identifying an animal in need of treatment for a pathogenic condition; determining a suitable target gene sequence for RNA interference that is aimed at inhibiting the growth of the pathogen; and contacting the animal with a dsRNA sequence that corresponds to the target gene sequence under conditions suitable for RNA interference, thereby treating the pathogenic condition.

Because siRNAs act as the primers for specific recognition of the RNA to be cleaved, there are structural features which have been identified to produce siRNAs which act most efficiently.

Many of the structural features of siRNAs have been identified and include a free 3′ hydroxyl group (this allows the siRNA to act as a primer for the RdRP reaction), a 5′ phosphate group, and 3′ overhangs. This most likely corresponds to the cleavage pattern of an RNase III-like enzyme. RNase III makes two staggered cuts in both strands of the dsRNA, leaving a 3′ overhang of 2 nucleotides. The “long” dsRNAs have been found to be processed by the cell into siRNAs. Thus, the large dsRNAs can be processed to 21-23 nucleotide siRNAs with a free 3′ hydroxyl group, a 5′ phosphate group, and 3′ overhangs of 2 nucleotides.

Synthetic dsRNAs, including siRNAs, can be synthesized using a variety of methods and when possessing the correct structural features, appear to work with high efficiency. Many of the useful structural features known in the art were identified using synthetic siRNAs. For example, siRNAs were produced that did not include a free 3′ hydroxyl group, they were considerably less efficient. However, a 5′ phosphate group did not appear to be necessary. When tested, 21 and 22 nucleotide RNA duplexes with 2 or 3 nucleotide overhanging 3′ ends were more efficient in reducing the target RNA expression than the corresponding blunt-ended dsRNAs or the dsRNA with 4 nucleotide overhangs. Thus, in one embodiment of the invention, double-stranded siRNAs are used directly to inhibit pathogen replication.

The structural features of the “long” double-stranded RNAs would appear to be less stringent since they are not active in the priming reaction but will simply be processed into the active siRNAs with the most advantageous features. However, overhangs of 17-20 nucleotides were less potent than blunt-ended siRNAs. The inhibitory effect of long 3′ ends was particularly pronounced for dsRNAs of less than 100 bp. Interestingly, a 5′ terminal phosphate, although present after dsRNA processing was not required to mediate target RNA cleavage and was absent from the short synthetic RNAs which worked with high efficiency. In addition, the size of the “long” double-stranded RNAs can have an effect on the efficiency.

Preferred lengths for efficient processing of dsRNA into 21 and 22 nucleotide fragments are determined by the fact that short dsRNA (<150 bp) appear to be less effective than longer dsRNAs in degrading target mRNA. However, 30 base pair dsRNAs were inefficiently processed to 21 and 22 nucleotide RNAs and were much less effective at mediating RNAi. Thus, “long” double-stranded RNAs can be from about 38 nucleotides to about full-length, from about 50 base pairs to about 1000 base pairs. The “long” double-stranded RNAs can range in size from about 150 base pairs to about 505 base pairs, including, but not limited to: 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, and 500 base pairs.

The target cleavage site was found to be located near the center of the region covered by the 21 or 22 nucleotide RNAs, 11 or 12 nucleotides downstream of the first nucleotide that is complementary to the 21 or 22 nucleotide guide sequence. Thus, it would be possible to design a pair of 21 or 22 nucleotide RNAs to cleave a target RNA at almost any given position. In addition, the overhangs did not need to be complementary to produce efficient cleavage. The direction of dsRNA processing determined whether a sense or an antisense target RNA was cleaved by the siRNP endonuclease. Certain chemical modifications (e.g., 2′-aminouridine, 2′-deoxythymidine, or 5-iodouridine) incorporated into dsRNA were well tolerated at the sense, but not the cleavage-guiding antisense strand.

To prepare a dsRNA useful in a method of the invention standard methods known in the art can be used as described, for example, in Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); and Dieffenbach and Dveksler, PCR Primer: A Laboratory Manual, Cold Spring Harbor Press (1995), all of which are incorporated herein by reference in their entirety. For example, RNA can be transcribed from PCR products, followed by gel purification. Standard procedures known in the art for in vitro transcription of RNA from PCR templates carrying, for example, T7 or SP6 promoter sequences can be used. The dsRNAs can be synthesized by using a PCR template and the Ambion (Austin, Tex.) T7 MegaScript kit, following the Manufacturer's recommendations and the RNA can then be precipitated with LiCl and resuspended in buffer. The specific dsRNAs produced can be tested for resistance to digestion by RNases A and T1. The dsRNAs can be produced with 3′ overhangs at both termini or one terminus of preferably 1-10 nucleotides, more preferably 1-3 nucleotides or with blunt ends at one or both termini. Thymidine nucleotide overhangs were found to be well-tolerated in mammalian cells, and the sequence of the overhang appears not to contribute to target recognition. Thus, any type of overhang can be used, however, the use of thymidine was found to reduce costs and can enhance nuclease resistance of siRNAs in the cell culture medium and within transfected cells.

Thus, a dsRNA, including siRNA, can be both partially or completely double-stranded. Generally, a siRNA encompasses to fragments of at least 18, at least 19, at least 20, at least 21, at least 22, at least 23, at least 24, at least 25, at least 30, at least 35, at least 40, at least 45, at least 50 or more nucleotides per strand, with characteristic 3′ overhangs of at least 1, at least 2, at least 3, or at least 4 nucleotides. As set forth above, a dsRNA can be of any length desired by the user as long as the ability to inhibit target gene expression is preserved.

The 21-23 nucleotide dsRNAs can be chemically synthesized by any method known to one of skill in the art, for example using Expedite RNA phosphoramidites and thymidine phosphoramidite (Proligo, Boulder, Colo.). Synthetic oligonucleotides can be deprotected and gel-purified. dsRNA annealing can be carried out by any method known in the art, for example: a phenol-chloroform extraction, followed by mixing equimolar concentrations of sense and antisense RNA (50 nM to 10 mM, depending on the length and amount available) and incubating in an appropriate buffer (such as 0.3 M NaOAc, pH 6) at 90° C. for 30 sec and then extracting with phenol/chloroform and chloroform. The resulting dsRNA can be precipitated with ethanol and dissolved in an appropriate buffer depending on the intended use of the dsRNA.

Two approaches can be used for expressing a double-stranded siRNA. In the first, the two nucleic acid sequence constituting the two strands of the RNA duplex are transcribed by individual promoters that drive their expression. In the second, the two strands of complementary nucleic acid sequences are expressed off a single promoter resulting in a fold-back stem-loop or hairpin structure that is processed into the dsRNA. A promoter useful in the present invention can be a promoter of eukaryotic or prokaryotic origin that can provide high levels of constitutive expression across a variety of cell types and will be sufficient to direct the transcription of a distally located sequence, which is a sequence linked to the 5′ end of the promoter sequence in a cell.

An inducible promoter is transcriptionally active when bound to a transcriptional activator that, in turn, is activated under a specific set of conditions, for example, in the presence of a particular combination of chemical signals that affect binding of the transcriptional activator to the inducible promoter and/or affect function of the transcriptional activator itself. Thus, an inducible promoter is a promoter that, either in the absence of an inducer, does not direct expression, or directs low levels of expression, of a nucleic acid sequence to which the inducible promoter is operably linked; or exhibits a low level of expression in the presence of a regulating factor that, when removed, allows high-level expression from the promoter, for example, the tet system. In the presence of an inducer, an inducible promoter directs transcription at an increased level. An inducible promoter is useful, for example, in prophylactic applications of the present invention such as the preparation of organisms transgenic for a dsRNA that can inhibit a pathogen upon induction of expression.

It is understood that the function of a promoter can be further modified, if desired, to include appropriate regulatory elements to provide for the desired level of expression or replication in the host cell. For example, appropriate promoter and enhancer elements can be chosen to provide for constitutive, inducible or cell type-specific expression. Useful constitutive promoter and enhancer elements for expression of a target gene transcript include, for example, RSV, CMV, CAG, SV40 and IgH elements. Other constitutive, inducible and cell type-specific regulatory elements are well known in the art. One skilled in the art will be able to select and/or modify the promoter that is most effective for the desired application and cell type so as to optimize target gene silencing resulting in pathogen inhibition.

Thus, promoters that are useful in the invention include those promoters that are sufficient to render promoter-dependent gene expression controllable for cell-type specificity, cell-stage specificity, or tissue-specificity, and those promoters that are inducible by external signals or agents. The promoter sequence can be one that does not occur in nature, so long as it functions in a vertebrate cell.

For the therapeutic and prophylactic applications of the present invention, the transient controllable expression of a dsRNA of the invention can allow for controlled pathogen inhibition. In this embodiment, the expression of the dsRNA transgene can be induced or suppressed by the simple administration or cessation of administration to an organism, respectively, of an exogenous inducer such as, for example, tetracycline or its derivative doxycycline. In this embodiment, the invention allows for efficient regulation of pathogen inhibition, a low background level of inhibition in the off state, fast induction kinetics, and large window of regulation by administering the inducer, for example, tetracycline or a tetracycline analogue to the individual. The level of dsRNA expression can be varied depending upon which particular inducer, for example, which tetracycline analogue is used. In addition, the level of dsRNA expression can also be modulated by adjusting the dose of the inducer that is administered to the patient to thereby adjust the concentration achieved in the circulation and in the tissues of interest. The inducer can be administered by any route appropriate for delivery of the particular inducing compound and preferred routes of administration can include oral administration, intravenous administration and topical administration.

There are several situations, for example, prophylactic applications, in which it may be desirable to be able to inhibit a pathogen at specific levels and/or times in a regulated manner, rather than simply inhibiting the pathogen constitutively at a set level. For example, a target gene can be silenced at fixed intervals to provide the most effective level of pathogen inhibition at the most effective time. As described herein, for a viral pathogen, inhibition can be most effectively accomplished late in the infection cycle rather than immediately upon entry of the viral genome into the host cell. The level of target gene product produced in a subject can be monitored by standard methods, for example, direct monitoring using an immunological assay such as ELISA or RIA or indirectly by monitoring of a laboratory parameter dependent upon the function of the gene product of interest, for example, blood glucose levels. The ability to effect pathogen inhibition at discrete time intervals in a subject allows for focused treatment of conditions only at times when treatment is necessary, for example, during the acute phase or during a particular stage of development.

A vector useful in the methods of the invention includes any nucleic acid that functions to carry, harbor or express the nucleic acid sequences corresponding to a dsRNA of the invention capable of inhibiting a pathogen. The structure of the vector can include any desired form that is feasible to make and desirable for a particular application of the invention. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

In certain embodiments, a viral vector can be used to practice the methods of the invention. As exemplified below, a dsRNA can be encoded on a retroviral vector, for example, a lentiviral vector. Unlike other retroviruses, lentiviruses have the ability to efficiently infect and transduce non-proliferating cells, including for example, terminally differentiated cells. Lentiviruses also have the ability to efficiently infect and transduce proliferating cells. Despite the pathogenesis associated with lentiviruses, it is well known to those skilled in the art that the undesirable properties of lentiviruses can be recombinantly separated so that its beneficial characteristics can be harnessed as a delivery vehicle for therapeutic or diagnostic nucleic acid sequences. Therefore, lentiviral-based vectors can be produced that are safe, replication-defective and self-inactivating, while still maintaining the beneficial ability to transduce non-dividing cells and integrate into the host chromosome for stable expression. A description of the various different modalities of lentiviral vector and packaging systems for vector assembly and gene delivery can be found in, for example, in Naldini et al., Science 272:263-267 (1996); Naldini et al., Proc. Natl. Acad. Sci. USA 93:11382-11388 (1996); Zufferey et al., Nature Bio. 15:871-875 (1997); Dull et al., J. Virol. 72:463-8471 (1998); Miyoshi et al., J. Virol. 72:8150-8157 (1998), and Zufferey et al., J. Virol. 72:9873-9880 (1998), all of which are incorporated herein by reference in their entirety.

Thus, in a therapeutic embodiment of the present invention a lentiviral vector can be useful for in vivo delivery and expression of a dsRNA corresponding to a target gene into both dividing and non-dividing cells. Methods for preparation of therapeutically safe third-generation lentiviral vectors are known in the art and include, for example, using only a fraction of the total genes normally present in the parent virus and ensures that the lentiviral vector is non-replicating. The genes that can be removed are genes associated with viral replication and pathogenesis, and their elimination is particularly important for the vectors derived from HIV. The removal of the viral replication and pathogenesis genes does not decrease the gene transfer efficiency of the lentiviral vector. If desired, the removal of these genes can be accompanied by the addition of a built-in self-inactivating safety feature that potentially eliminates the possibility that the vector could replicate or recombine with infectious virus during vector manufacturing or patient treatment.

Briefly, to generate a lentiviral vector useful for practicing the methods of the invention for inhibiting a pathogen, all of the viral genome can be removed from the virus and replaced by the dsRNA sequences. The essential cis-acting sequences, such as the packaging signal sequences, which are required for encapsidation of the vector RNA, can be included in the vector construct. The viral sequences necessary for reverse transcription of the vector RNA and integration of the proviral DNA, the LTRs, the transfer RNA-primer binding site, and the polypurine tract (PPT) can be incorporated into a lentiviral vector of the invention. If desired, further modifications known in the art and described herein can be introduced into a lentiviral vector production system, for example, to effect an increase in viral titers. Lentiviral vector systems useful in the invention that incorporates a third-generation, Tat-free packaging system are wel known in the art and described, for example, by Dull et al., Journal of Virology 72:8463-8471 (1998); Pfeifer et al., Procl. Natl. Acad. Sci. USA 97:12227-12232 (2000), which are incorporated herein by reference in their entirety.

An HIV-derived vector system useful in the invention can consist of at least two or more, three or more, four or more separate transcriptional units, which can be located, for example, on separate nucleic acid constructs. The Tat, which serves as a transactivator of the LTR, can be omitted in this system if part of the upstream LTR in the transfer vector is replaced by constitutively active internal promoter sequences, for example, CMV or CAG. Furthermore, expression of rev in trans can be sufficient with a plasmid that contains only gag and pol coding sequences from HIV. If desired, the first vector component of a lentiviral vector production system of the invention can contain the lentiviral gag, pol and rev genes on one or more separate nucleic acid molecules, for example, plasmids. If rev is deleted from the transfer component of the vector production system, it is necessary to provide the transfer vector and packaging vector with cis acting sequences that replace Rev/RRE function.

Furthermore, the transfer vector component of a vector production system of the invention can incorporate a self-inactivating (SIN) LTR rendering the vector itself self-inactivating due to a deletion in a region at the end of the virus genome called the long-terminal repeat (LTR), which describes unique cis-acting sequences that flank the virus genome and are essential to the virus life cycle. A sequence within the upstream LTR serves as a promoter under which the viral genome is expressed. Briefly, the U3 region of the 3′LTR, which harbors the major transcriptional functions of the lentiviral genome, can be deleted. During the process of reverse transcription, the 3′LTR is copied to the 5′LTR. By deleting non-replicative portions of the 3′LTR, the genomic viral DNA is inserted into the target genome as a promoter-less sequence. Inactivation of the promoter activity of the LTR can serve as an important safety feature of the vectors of the invention since it reduces the possibility of insertional mutagenesis.

As described herein, other modifications to enhance safety and specificity include the use of specific internal promoters that regulate gene expression, either temporally or with tissue or cell specificity as well as the introduction of post-transcriptional regulatory elements that enhance expression of the dsRNA including, for example, the Woodchuck hepatitis virus post-transcriptional regulatory element (WPRE) and the Cana PPT flap, as described, for example, by Zephyr et al., J Viol. 1999. 73(4):2886-92; Zennou et al., Cell 101:173-85 (2000), both of which are incorporated herein by reference.

If desired, a retroviral vector useful in the invention can further be pseudotyped to increase host range. In this embodiment, the retroviral env gene can be deleted from the packaging component of the vector system and instead the envelope gene of a different virus can be supplied on a third component. As has been extensively described in the art, a commonly used envelope gene is that encoding the G glycoprotein of the vesicular stomatitis virus (VSV-G), which confers stability to the particle and permits the vector to be concentrated to high titers.

Packaging cell lines for vector poduction can be chosen that continuously produce high-titer vector. A packaging cell line useful for producing a retroviral vector of the invention further can be one in which the expression of packaging genes and VSV-G, and therefore the production of vector, can be turned on at will as described by Kafri et al., J. Virol. 73(1): 576-84 (1999), which is incorporated herein by reference.

A pseudotyped viral vector that encodes a dsRNA capable of inhibiting a pathogen can be produced by transfecting cells with a viral vector, for example, a retroviral vector. As described herein, exemplary host cells for transfection with the lentiviral vector production system include, for example, mammalian primary cells; established mammalian cell lines, such as COS, CHO, HeLa, NIH3T3, 293T and PC12 cells; amphibian cells, such as Xenopus embryos and oocytes; and other vertebrate cells. Exemplary host cells also include insect cells (for example, Drosophila), yeast cells (for example, S. cerevisiae, S. pombe, or Pichia pastoris) and prokaryotic cells (for example, E. coli).

Methods for introducing a nucleic acid into a host cell are well known in the art and include, for example, various methods of transfection such as calcium phosphate, DEAE-dextran and lipofection methods, electroporation and microinjection. The methods of isolating, cloning and expressing nucleic acid molecules of the invention referred to herein are routine in the art and are described in detail, for example, in Sambrook et al., Molecular Cloning: A Laboratory Manual, Cold Spring Harbor Laboratory, New York (1992) and in Ausubel et al., Current Protocols in Molecular Biology, John Wiley and Sons, Baltimore, Md. (1998), which are incorporated herein by reference. With particular regard to preparation of a dsRNA corresponding to a target gene, it is understood by those skilled in the art that sequence verification of the dsRNA templates after cloning is useful, since even a single nucleotide mismatch between the target gene's mRNA and the dsRNA antisense strand component of the double-stranded dsRNA can reduce or prevent inhibition.

In providing a patient (or cell) with the dsRNAs, the dosage of administered agent will vary depending on such factors as the patient's age, weight, height, sex, general medical condition, previous medical history, and the like. In addition, the dosage will vary depending on the pathogen and the method of treatment. For example, if dsRNA is to be administered to a patient having a systemic infection, more will be needed then if the infection is more localized. In addition, a targeted vector can allow administration of a lower dosage. The dsRNA can be administered using vectors or can be administered as “naked” DNA. Alternatively, the dsRNA can be attached to a carrier protein or moiety such as a bead. One vector useful for in vivo and ex vivo delivery is a liposome or comparable vesicle-like structure. The liposome can be produced in a solution containing the dsRNA so that the dsRNA is encapsulated during polymerization. Alternatively, the liposomes can be polymerized first, and the dsRNA can be added later by resuspending the polymerized liposomes in a solution of dsRNA and treating with sonication to effect encapsulation. In one embodiment, the liposome is produced so that in the right pH or under the right conditions, the dsRNA is evulsed. For example, “micromachines” evulse their contents when treated with a specific frequency radio wave. Alternatively the liposomes can be produced to be uncharged which will allow them to be taken up by the cell.

Two approaches can be used for expressing a double-stranded RNA. In the first, the nucleic acid sequences constituting the dsRNA are transcribed by individual promoters that drive their expression. In the second, the complementary nucleic acid sequences are expressed off a single promoter resulting in a fold-back stem-loop or hairpin structure that is processed into the siRNA. Where driven off a single promoter, the two nucleic acid sequences making up the dsRNA upon expression are the reverse complement of one another so as to result in complementarity upon fold-over into the hairpin structure. Alternatively, where the nucleic acid sequences are not expressed from a single transcriptional unit and, consequently, do not fold over into a hairpin structure the two nucleic acid sequences making up the dsRNA are complementary in sequence as dictated by base-pairing.

For ex vivo applications of the invention cells from the mammal or pathogen can be isolated and contacted with the siRNAs or the “long” double-stranded RNAs. The dsRNAs can be induced to be taken up by the cells using any method known to one of skill in the art, including but not limited to transfection, transformation, lipofection, electroporation, microinjection, transduction, infection, use of viral vectors, and using products such as TansMessenger Transfection Reagent™, PolyFect transfection reagent™, Effectene transfection reagent™, and SuperFect™ (all from Qiagen, Inc.), and Lipofectamine™ (Gibco). The cells are then re-introduced into the mammal.

Ex vivo gene therapy methods can be useful for practicing the therapeutic applications of the invention. For example, cells can be removed from the patient, for example, an HIV-infected patient. Briefly, bone marrow cells can removed, cultured in vitro and subsequently infected with, for example, a lentiviral vector engineered to encode a stem-loop RNA that would be processed by the Dicer nuclease to yield an effective siRNA molecule. In this example, the siRNA would be complementary to a region of the HIV genome. As an example of a lentiviral vector, the self-inactivating (SIN) HIV vector (p156RRLsinPPTCMVGFPPRE) can be used. The SIN HIV-based vector supernatants can be prepared by three-plasmid cotransfection into 293T cells with pVSVG, pdeltaR9, and p156RRLsinPPTCMVGFPPRE. Typical vector stocks are about 3×10₇ infectious units per ml. To achieve infection of 10⁸ human cells, the lentiviral vector particles can be concentrated to 10⁸-10⁹ infectious units per ml and one ml applied to the cultured bone marrow cells. Cells can then be reintroduced into the patient. This procedure can provide a supply of cells impervious to HIV infection. Moreover, RNAi in model organisms is known to spread away from its site of action, so this ex vivo gene therapy treatment can also nucleate the spread of the effect throughout the patient.

For in vivo gene therapy, any methods of known in the art can be used. In addition, any gene therapy vector can be used to produce the dsRNA, for example, by encoding an RNA hairpin. Many such vectors are easily obtainable from commercial vendors known to those skilled in the art. However, in one implementation of gene therapy, a replicating virus can be engineered to contain (RNA virus) or produce (DNA virus) an RNA precursor of the desired siRNA. For example, a replication competent vaccinia virus can be used, which is engineered to encode an RNA hairpin which is subsequently converted into an siRNA. Alternatively, an RNA virus such a picorna virus can be engineered to contain an RNA hairpin as a part of its genome. In either case, the RNA structure can be designed so that the hairpin could be cleaved by Dicer or other nuclease to produce the siRNA. Replication of the virus would thereby seed many tissues with the siRNA.

Inoculation with siRNA is another application of the invention that exploits the ability of RNAi to spread from the site of initial infection. If desired, siRNAs can be introduced by “GeneGun” as in typical DNA-mediated vaccination. siRNAs can be affixed to beads, and beads ballistically introduced into muscle using the Gene Gun. RNAi can be initiated at the site of injection, then spread systemically, as has been observed in worms and plants. As an alternative, DNAs can be introduced that encode hairpin structure RNAs in front of a promoter active in human cells. Introduction of the DNA into human cells can be accomplished by GeneGun, injection, or other known methods. Transcription would yield the hairpin RNA, which can then be cleaved by Dicer or other nuclease to yield the siRNA.

In worms, soaking the animal in siRNA has been shown to initiate RNA interference. Thus simple soaking of tissue with RNA can be used to introduce the RNAi effect. For example, a mammalian embryo can be treated by addition of siRNA to the amniotic fluid.

The RNAi effect also can be induced by feeding nucleic acid to worms. In this implementation, siRNA or DNA encoding siRNA can be fed to patients in therapeutically effective doses. This would then induce the RNAi effect. As appropriate, nucleic acids can be formulated to protect them chemically and promote uptake in the human gastrointestinal tract.

Viral vectors can be produced to target a specific cell type, genus, species, or even to target or to be expressed specifically in infected cells. For example, muscle-specific or lung-specific promoters are obtainable, allowing directed expression of the siRNA in that tissue. Alternatively, the virus can be engineered to contain a ligand which targets a specific cellular receptor and can even be engineered to target only infected cells. These methods are known to the skilled artisan.

Transfection of siRNAs will vary depending on in vivo or ex vivo methods as well as the vectors that are used. Ex vivo methods can use concentrations of dsRNA from about 25nM to about 1.5 nM siRNA duplexes with respect to the final volume of tissue culture medium. Increasing the concentration to 100 nM is not envisioned to enhance transformation because it can affect transformation efficiencies.

If desired, the dsRNA can be administered to an organism by subcutaneous, intravascular, or intraperitoneal injection. If desired, a slow-release device, such as an implantable pump, can be used to facilitate delivery of a dsRNA to cells of the organism. A particular cell type within an orrganism can be targeted by modulating the amount of the dsRNA administered and by controlling the method of delivery. For example, intravascular administration to the portal, splenic, or mesenteric veins or to the hepatic artery of a mammal can be used to facilitate targeting the dsRNA to liver cells. In another method, the dsRNA can be administered to cells or organ of a donor individual (human or non-human) prior to transplantation of the cells or organ to a recipient.

In a preferred method of administration, the dsRNA is administered to a tissue or organ containing the targeted cells of the organism. Such administration can be accomplished by injecting a solution containing the lentiviral vector of the invention into a tissue, such as skin, brain (e.g., the olfactory bulb), kidney, bladder, trachea, liver, spleen, muscle, thyroid, thymus, lung, or colon tissue. Alternatively, or in addition, administration can be accomplished by perfusing an organ or an entire organism with a solution containing the dsRNA, according to conventional perfusion protocols. Alternatively, a gene gun can be used to localize the treatment. Depending on the mode and vector, the dsRNA or vector can be introduced within any buffer, additive, excipient, or pharmaceutically acceptable solution.

Depending on the disease which is being treated, combining the invention methods for inhibiting a pathogen with other treatments can be advantageous. For example, the dsRNAs can be administered in combination with antibiotics, immune suppressors, immune activators, pain medications or anesthetics, anti-inflammatories, antivirals, chemotherapeutics, anti-fungals, etc.

Several vector-based methods can be utilized to allow the dsRNA of interest to be expressed inside the target organism. In some of these methods, a recombinant nucleic acid sequence construct is prepared, linking the nucleic acid that encodes the dsRNA sequence to regulatory sequences and vehicles that allow transfer and/or expression of the sequence. The term construct refers to a recombinant nucleic acid sequence, generally a recombinant DNA molecule, that has been generated for the purpose of the expression of a specific nucleotide sequence(s), or is to be used in the construction of other recombinant nucleotide sequences. The construct can be generated for the purpose of controlling the expression of a specific nucleotide sequence(s) as, for example, in a construct containing a viral enhancer.

As used herein, the term “vector” is used in reference to nucleic acid molecules that transfer nuclear acid segment(s) from one cell to another. Vectors are used to introduce a nuclear acid molecule into a host cell where it can be replicated (i.e., reproduced) in large quantities. Vectors, including cloning vectors allow the insertion of nuclear acid sequences without the loss of the vector's capacity for self-replication. Cloning vectors can be derived from viruses, plasmids or genetic elements from eucaryotic and/or procaryotic organisms; vectors frequently comprise DNA segments from several sources. Examples of cloning vectors include plasmids, cosmids, lambda phage vectors, P1 vectors, yeast artificial chromosomes (YACs), and bacterial artificial chromosomes (BACs).

The dsRNA, including the siRNA, sequences of the present invention can be expressed in vitro by transfer of the sequences into a suitable host cell. “Host cells” are cells in to which a vector containing a nuclear acid molecule is introduced. The term also includes any progeny or graft material, for example, of the subject host cell. It is understood that all progeny can not be identical to the parental cell since there can be mutations that occur during replication. However, such progeny are included when the term “host cell” is used. Methods of stable transfer, meaning that the foreign DNA is continuously maintained in the host, are known in the art.

The RNAi-inducing nucleic acid sequences according to the present invention can be inserted into a recombinant expression vector. The terms “recombinant expression vector” or “expression vector” refer to a plasmid, virus or other vehicle known in the art that has been manipulated by insertion or incorporation of the genetic sequence. Such expression vectors contain a promoter sequence which facilitates the efficient transcription of the inserted RNAi sequence. The expression vector typically contains an origin of replication, a promoter, and one or more genes that allow phenotypic selection of the transformed cells.

Methods well known to those skilled in the art can be used to construct expression vectors containing the RNAi-inducing nucleic acid sequence and appropriate transcriptional/translational control signals. These methods include in vitro recombinant DNA techniques, synthetic techniques, and in vivo recombination/genetic techniques.

A variety of host-expression vector systems can be utilized to express the dsRNA and siRNA sequences in numerous types of organisms. These include, but are not limited to, microorganisms such as bacteria transformed with recombinant bacteriophage DNA, plasmid DNA or cosmid DNA expression vectors containing the sequence; yeast transformed with recombinant yeast expression vectors containing the sequence; insect cell systems infected with recombinant virus expression vectors (e.g., baculovirus) containing the sequence; or animal cell systems infected with recombinant virus expression vectors (e.g., retroviruses, adenovirus, vaccinia virus) containing the siRNA or dsRNA-encoding sequences, or transformed animal cell systems engineered for stable expression.

Transcriptional regulatory sequences are nuclear acid sequences such as initiation signals, enhancers, and promoters, which induce or control transcription of a gene or genes with which they are operably linked. In designing the method of transfer of RNAi-inducing nucleic acid to the host, one can link the nucleic acid encoding the RNAi-inducing sequence to a transcriptional regulatory sequence which allows constitutive expression. Alternatively, the transcriptional regulatory sequence can allow inducible expression, environmentally-regulated expression, or developmentally- regulated expression.

Any of a number of suitable transcription and translation elements, including constitutive and inducible promoters, transcription enhancer elements, and/or transcription terminators, can be used in the expression vector (see e.g., Bitter, et al., Methods in Enzymology 153:516, (1987)). The choice of these elements will vary depending on the host/vector system utilized. The particular promoter selected should be capable of causing sufficient expression to result in the production of an effective amount of siRNA or dsRNA gene product. The promoters used in the vector constructs of the present invention can be modified, if desired, to affect their control characteristics.

For example, when cloning in bacterial systems, inducible promoters such as pL of bacteriophage, plac, ptrp, ptac (ptrp-lac hybrid promoter) and the like can be used. When cloning in mammalian cell systems, promoters derived from the genome of mammalian cells (e.g., metallothionein promoter) or from mammalian viruses (e.g., the retrovirus long terminal repeat; the adenovirus late promoter; the vaccinia virus 7.5K promoter) can be used. Promoters produced by recombinant DNA or synthetic techniques can also be used to provide for transcription of the inserted RNAi-inducing sequence.

The term “transfection” refers to a process for introducing heterologous nucleic acid into a host cell or organism. A transfected cell refers to a host cell, such as a eukaryotic cell, and more specifically, a mammalian cell, into which a heterologous nucleic acid molecule has been introduced. The nucleic acid molecule can be stably integrated into the genome of the host or the nucleic acid molecule and can also be present as an extrachromosomal molecule, such as a vector or plasmid. Such an extrachromosomal molecule can be auto-replicating.

Transfection therefore refers to the insertion of an exogenous nucleic acid into a host cell, irrespective of the method used for the insertion, for example, direct uptake, transduction, mating or electroporation. The terms “host cells” and “recombinant host cells” are used interchangeably herein. It is understood that such terms refer not only to the particular subject cell but to the progeny or potential progeny of such a cell. Because certain modifications can occur in succeeding generations due to either mutation or environmental influences, such progeny may not, in fact, be identical to the parent-cell, but are still included within the scope of the term as used herein.

The methods and small or long RNAs can be used to treat any disease which is caused by a pathogen. RNAi is expected to have a very broad effect. For example, treatment of an organism using RNAi will spread throughout the organism, depending on the size of the organism and the number of cells initially effected. Initiation of RNAi rquires a cell which contains the correct cellular machinery. Thus, the method is expected to work on any pathogens which contain the machinery for RNAi, as well as any pathogens which infect a cell or are otherwise internalized by a cell which possesses the machinery for RNAi. This can include organisms which are phagocytized within the body and the phagocytosis can be enough to have an effect on pathogens which are extracellular. Thus, it is envisioned that although intracellular pathogens are optimally effected by the treatment, RNAi can also affect extracellular pathogens, and will likely affect pathogens which possess the machinery for carrying out RNAi.

In addition, it is envisioned that the method of administration and the genes which are targeted can vary depending on the pathogen and its method of pathogenesis. For example, intracellular pathogens are best treated using methods which result in the siRNAs being produced or localized within a human or mammalian cell, particularly in an infected mammalian cell. Extracellular pathogens which possess the RNAi machinery can best be treated using methods which result in the siRNAs being produced or localized within the pathogen itself.

Intracellular pathogens include genomic elements and all obligate intracellular parasites, including but not limited to: all viruses, mycoplasma, mycobacteria, chlamidia, rickettsia, and plasmodium. Intracellular pathogens can also include pathogens which only carry out a part of their lifecycle within cells, but can survive outside of cells for a certain period of time. In addition, any pathogens which are phagocytized can be effectively treated with RNAi.

Genomic parasites, including, but not limited to, transposons such as LINEs, SINEs, LTR retrotransposons, DNA transposons can also be treated with RNA interference. In a further embodiment, pathogenic human viruses, including, but not limited to, picornaviruses, caliciviruses, togaviruses, flaviviruses, coronaviruses, rhabdoviruses, filoviruses, paramyxoviruses, orthomyxoviruses, bunyaviruses, arenaviruses, reoviruses, birnaviruses, retroviruses, lentiviruses, hepadnaviruses, parvoviruses, papovaviruses, adenoviruses, herpesviruses, poxviruses, iridoviruses can be treated with the method and dsRNAs. In a further embodiment, intracellular pathogenic bacteria, including, but not limited to, mycobacteria, mycoplasma, and rickettsias can be treated using RNA interference.

Other types of bacteria, including, but not limited to, gram negative, gram positive, spirochetes, vibrio, and other eubacteria can be treated with RNA interference by targeting the phagocytosis.

Eukaryotic human parasites, including, but not limited to, fungii, helminths, plasmodia (and other members of the apicomplexans), trypanosomes, schistosomes, hookworm, tapeworm, and amoeba can be treated using the dsRNAs and methods herein, particularly those eukaryotes which possess the machinery for RNAi and those which are intracellular.

A target gene can be any gene that is present and expressed in the pathogen or the host cell, provided that at least such part of the target gene sequence is known as is sufficient to allow selection of the nucleic acid sequence of the dsRNA corresponding to the target gene. Thus, it is not required that the entire sequence of the target gene is known to the user practicing the invention.

The nucleic acid sequence for a dsRNA of the invention that corresponds to a target gene can be selected based on a variety of considerations. To select the nucleic acid sequence either part of or the entire target gene sequence can be scanned and potential sequence sites can be recorded. Potential sequence sites can then be evaluated by a BLAST analysis against the GENBANK database to disqualify any target sequence with significant homology to other genes. Furthermore, dsRNAs can be designed to regions of target mRNA with low secondary structure. If desired, two or more nucleic acid sequences can be selected for preparation of separate transfer vehicles capable of inhibition of a pathogen. This approach allows for comparison of the efficiency of pathogen inhibition between the nucleic acid sequences representing the target gene.

Thus, the dsRNAs can target any gene desired by the user. The target gene can be a host cellular gene as well as a gene from the pathogen, for example, a viral gene. Preferably, the targeted genes are essential genes such that, when no longer translated, the pathogen will die or be unable to replicate. However, it is envisioned that RNA viruses as well as viruses which produce a single multigenic mRNA can be affected using any sequence within the genome, including nonessential genes and non-coding regions. Examples of preferred target genes for various pathogens follow. It is understood that these are merely provided as exemplary and are not meant to narrow the target gene choice. For example, HIV genes which would be particularly advantageous to target include gag, pol (including int), and env. Genes best targeted for DNA viruses are those that encode the capsid proteins, any polymerases which they carry and require, any required DNA binding proteins, and any genes required to evade host-cell defenses. It is envisioned that siRNAs against the majority of RNA viruses will work when targeted to any sequence in the viral genome. In fact, non-coding regions can even be targeted because amplification of RNAi would cause the effect to spread to required genes, inactivating them as well. However, it is likely that sequences which are more internal within the genome would be best targeted and that by targeting essential genes, a stronger, faster clearance of the virus or pathogen can be effected.

Genes targeted for intracellular and extracellular bacteria include any essential genes which are not complementary to genes within the host cell's repertoire. For example, genes which are involved in unusual energy sources, genes which target the bacterial ribosome, genes which are enzymes specific to the bacterium which are necessary for growth such as bacterial RNA and DNA polymerases, genes which are essential for binary fission, and genes that are essential for bacterial genome integrity.

Eukaryotic pathogens are more quickly and effectively treated by targeting essential genes which are specific to the eukaryote and are not complementary to host genes. Examples include genes which code for enzymes which control the process of budding in yeast, eukaryotic RNA and DNA polymerases, etc. These genes can vary depending on the type of organism. The vectors, siRNAs or dsRNAs can be administered using any method known to one of skill in the art. The following examples of methods of administration are included in some detail and can be particularly advantageous.

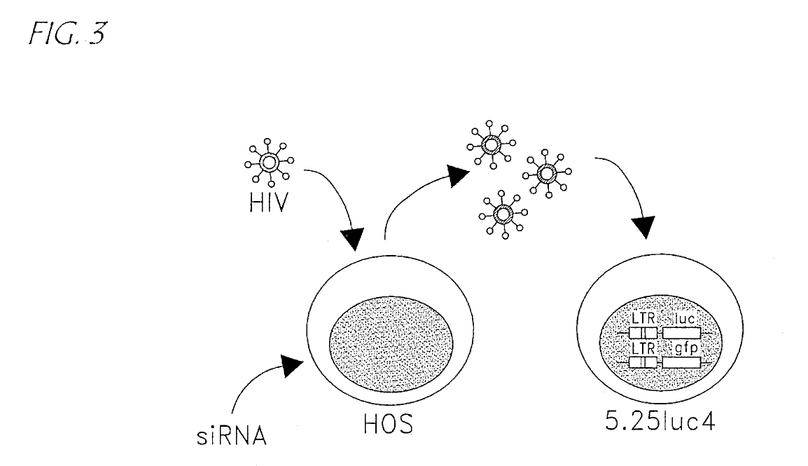

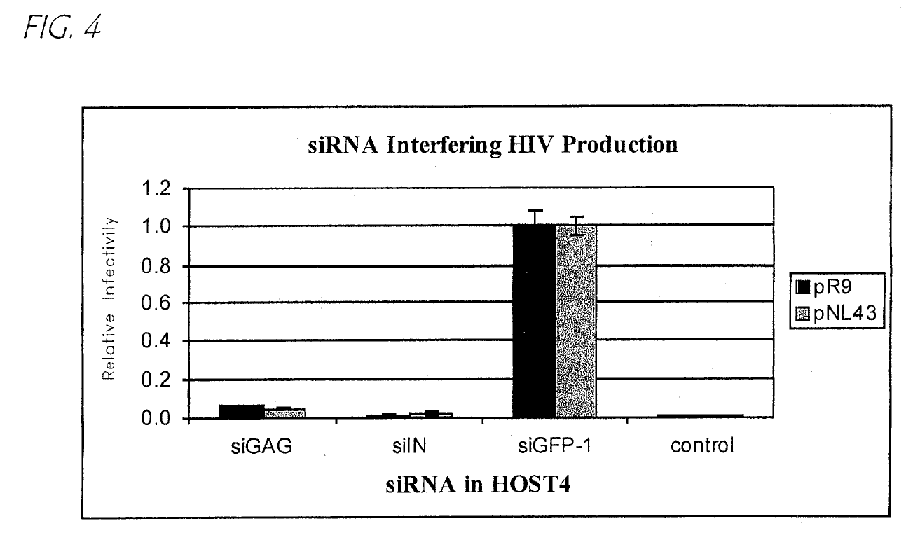

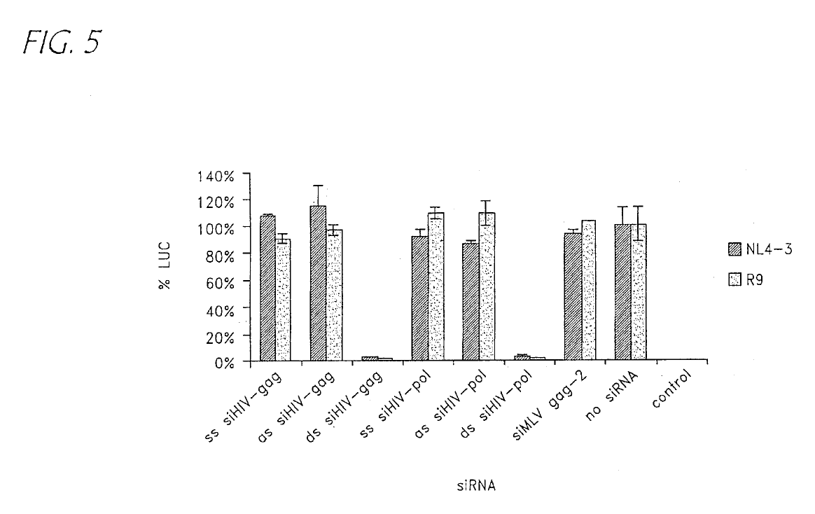

An HIV model of infection of human cells was used to investigate whether RNAi would block pathogenic infection of a mammalian cell. To determine whether RNAi can be active against a human virus using HIV as a model system, a cell culture system that is i) readily infectable by HIV, ii) easily transfected to allow introduction of RNAs, and iii) inducible for RNAi was identified. Human cells that were susceptible to infection by HIV, and supported a robust RNAi response, were identified in Example I. A schematic diagram of the method is shown in FIG. 3. Briefly, HOS.T4.CXCR4 cells were transfected with siRNAs and were then infected with HIV-1 2 hr later. Two days later, culture supernatants were harvested and applied to 5.25luc4 indicator cells to determine the extent of RNAi-based inhibition. Integration of HIV-1 cDNA followed by synthesis of Tat protein activates the production of luciferase and green fluorescent protein from the integrated HIV-1 LTR-luc and LTR-gfp reporter genes. The results (see FIGS. 4 and 5) show that transfection of siRNAs against the HIV gag or integrase coding regions inhibited HIV replication >95%. Transfection of an siRNA against the unrelated GFP gene did not block HIV replication, indicating the specificity of inhibition. These data provide one example of RNAi activity against a human pathogen, establishing that RNAi can be considered a branch of the human adaptive immune system that is programmed by RNA instead of antigen. RNAi-mediated inhibition decayed with a half-life of about a week. These data indicate that RNAi has the potential be used therapeutically to obstruct replication of human pathogens.

In ovo electroporation of chick embryos is a useful and convenient system for testing RNAi in vertebrates. In this method, RNAi targeted against RSV was found to inhibit retroviral replication and pathogenesis. RNAi was also found to block retroviral replication in cell culture. This method is demonstrated in Examples 7 through 11, below. Nucleic acids were injected into a chicken embryo neural tube 2 days after fertilization, then a current was applied in an orthogonal direction. This introduced the RNAi nucleic acid into cells near the positive electrode. In initial experiments, it was demonstrated that siRNAs against the gene for green fluorescent protein (GFP) could inhibit expression of GFP. Subsequently, it was determined whether RNAi could inhibit the replication of a nonpathogenic derivative of Rous sarcoma virus (RSV). Plasmid DNAs encoding the RSV genome were electroporated into embryos along with siRNAs against RSV or control siRNAs, and this allowed for the demonstration of RNAi-mediated control of RSV infection. Electroporation of pathogenic RSV resulted in disruption of the neural tube and death of embryos, but this could be reversed by coelectroporation of RNAi. Viral RNA genomes just entering cells were insensitive to RNAi, but, late during infection, the viral genomes were efficiently degraded. Together these data establish that the RNAi system would control the replication of retroviruses and begin to specify the inhibitory mechanism.

Thus, RNAi can act as an effective antiviral system in vertebrates. These findings provide a convenient vertebrate animal model for studies of RNAi, and they open a wide range of possibilities for the use of RNAi against viral diseases.

The method and compositions described herein can be used to treat any animal, including for veterinary purposes. The animal species can be vertebrates or invertebrates. The invertebrates can be insects or crustaceans. The vertebrates can be selected from mammals, birds, amphibians, reptiles, or fish. In addition, it is clear that such a method would be particularly useful for those patients who are immunocompromised in any way, including the elderly, the very young, those with autoimmune diseases or receiving transplants, and those with diseases which target the classic immune system. It is envisioned that the treatment can be used for infections which occur in utero, including, but not limited to, HIV, HSV, toxoplasmosis, syphilis, and German measles. In this case, the method of administration can involve injection into the uterine sac. It is likely that dsRNAs would then be taken up by the fetus and RNAi induced.

It is also envisioned that the method can be used in tissue culture, in cells which are part of an organ to be transplanted, in eggs, sperm, or any-cell which is to be injected or transplanted into a new host. Any pathogens or parasites which such cells and organs are harboring can be removed before transplantation.

The demonstration of RNAi activity against retroviral pathogenesis suggests diverse possible applications in the prevention and therapy of disease. Practical application of RNAi technology will be facilitated by the use of engineered DNAs containing inverted repeat sequences, which can produce hairpin RNAs that are processed by the dicer nuclease to yield active siRNAs. If such molecules are well tolerated by cells, it can be possible to stably incorporate siRNA-producing DNA molecules in vertebrate cells to inhibit viral replication and pathogenesis.

The invention also provides a method of making a transgenic organism capable of expressing a dsRNA that corresponds to a target gene in a pathogen, said method by the steps of identifying a target gene in the pathogen; preparing a nucleic acid sequence having a region that corresponds to a portion of the target gene, wherein the nucleic acid is able to form a double-stranded RNA once expressed in the animal; contacting a recipient animal with the nucleic acid; producing one or more offspring of the recipient animal; and testing the offspring for expression of the double-stranded RNA.

The nucleic acid can be contained on a vector that can be chosen based on a variety of criteria and can be any nucleic acid molecule capable of transferring another nucleic acid sequence to which it has been linked,, for example, a virus, plasmid, cosmid or transposon. If desired, the nucleic acid also can be administered in “naked” form.

The recipient organism can a pre-implantation mammalian embryo that is subsequently transferred into a pseudo-pregnant female. If so, the method of the invention can further involve the step of allowing the embryo to develop into at least one viable transgenic mammal in which the expression of the target gene is inhibited by the presence of the double-stranded target gene transcript.

The recipient organism can also be a bird, for example, a chicken or turkey. In this embodiment of the invention, primordial germ cells or their precursors can be transfected with the nucleic acid corresponding to the target gene in culture. Once transfected, the cells can then be used to contact the recipient animal, for example, by microinjection. The recipient organism will represent a germline chimera and can subsequently be bred and the offspring screened for expression of the dsRNA and, if indicated, inhibition of the pathogen. For prophylactic applications, the nucleic acid sequence can be expressed from an inducible promoter. This embodiment of the invention allows for breeding livestock that is genetically altered to resist pathogenic infections.

For general discussions of transgenic methods suitable for farm animals, see, for example, Montoliu, (2002) Cloning Stem Cells 4:39-46; Wheeler and Walters, (2001) Theriogenology 56:1345-1369; and Wolf et al. (2000), Exp. Physiol. 85:615-625, all of which are herein incorporated by reference in their entireties. Transgenic swine with stable incorporation of the desired nucleic acid sequences can be produced, for example, following the method of Wall et al., (1991) Proc. Natl. Acad. Sci. USA 88:1696-1700, and Velander et al., (1992) Proc. Natl. Acad. Sci. USA 89:12003-12007, both of which are incorporated by reference in their entireties. Methods to produce transgenic cattle are provided, for example, in Chan et al. (1998), Proc. Natl. Acad. Sci. USA., 95:14028-14033, which is incorporated herein by reference in its entirety. Types of mammals that can be treated using the method of the invention include but are not limited to bats, beefalo, boar, buffalo, cats, cattle, chimpanzees, cows, deer, dogs, donkeys, elk, fox, goats, guinea pigs, horses, humans, llamas, mice, monkeys, mules, pigs, rabbits, rats, reindeer, sheep and water buffalo.

Thus, the invention also provides a transgenic organism produced by the methods described herein. In particular embodiment, the transgenic organism prepared by the methods described herein is a vertebrate, for example, a mammal or a bird. The ability to prepare a organism transgenic for a nucleic acid sequence capable of inhibiting a pathogen can be useful for therapeutic and prophylactic applications of the invention where pathogen resistance can be achieved.

Exemplary diseases of cattle include blackleg, tetanus, lungworm disease, rotavirus, infectious bovine rhinotracheictis, respiratory syncytial virus, pasteurellosis, enteritis, leptospirosis, mastitis, ringworm, coronavirus, salmonella, and E Coli. Exemplary diseases of sheep and goats include clostridial diseases such as tetanus, pasteurellosis, chlamydiosis, toxoplasmosis, louping ill, contagious pustular dermatitis, and footrot. Exemplary diseases of pigs include erysipelas, parvovirus, colibacillosis, clostridial disease, atrophic rhinitis, enteritis, and porcine pneumonia. Examplary diseases of fish which can be treated by the method of the invention include enteric redmouth disease, furunculosis, and vibriosis.

One particularly useful application of RNAi is that farm animals might be engineered to resist economically important infections. For example, several viral diseases of chickens might be targeted, including fowl pox, chicken flu virus, chicken arjemia virus and ALV-J (avian leukosis virus, subtype J). The findings shown herein that RNAi is highly active in chicken embryos suggests that it can be possible to block the replication of these viruses by introducing genes producing inhibitory siRNAs into the chicken germline. Many other applications in animal husbandry can be also envisioned.

Types of infectious diseases in birds which can be treated by the method of the invention include but are not limited to viral, bacterial, fungal, and protozoan diseases. Viral diseases which can be treated by the method of the invention include but are not limited to avian reovirus, West Nile Virus, avian encephalomyelitis, chicken anaemia virus, duck virus enteritis, erysipelas, pacheco's disease, psittacine beak and feather disease, psittacine wasting disease, avian infectious bursal disease, pox virus, polyoma virus, egg drop syndrome, newcastle disease, mareks disease, fowl pox, infectious laryngo-tracheitis, and infectious bronchitis.

Bacterial diseases of birds which can be treated by the method of the invention include but are not limited to Chlamydiosis, Pullorum, Chronic Respiratory Disease (CRD) Coryza, (Hemophilus paragallinarium), Fowl Cholera, (Pasteurella multocida), salmonellosis, Avian mycoplasmosis, (caused principly by three species: Mycoplasma gallisepticum, Mycoplasma synoviae, and Mycoplasma meleagridis).

Fungal infections of birds, such as Aspergillosis, and Candida can also be treated by the method of the invention. Protozoan diseases include but are not limited to such diseases as Coccidiosis, Blackhead (Histomoniasis, Enterohepatitis) caused by a protozoan parasite called Histomonas meleagridis, and Hexamitiasis (Infectious Catarrhal Enteritis).

Types of birds that can be treated using the method of the invention include but are not limited to: Albatross, Anhingas, Anis, Apostlebirds, Asities, Auklets, Avocets, Babblers, Barbets, Barn Owls, Bee Eaters, Bell Birds, Birds of Paradise, Bittern, Blackbirds, Bluebirds, Bluethroats, Bobolinks, Bobwhites, Boobies, Bowerbirds, Broadbills, Budgies, Bulbuls, Bunting, Bushtits, Canaries, Caracaras, Cardinals, Cassowaries, Catbirds, Chachalacas, Chats, Chickadees, Chickens, Cisticolas, Cockatoos, Condors, Coots, Cuckoos, Cuckoo-Rollers, Curassows, Curlews, Cormorants, Corncrake, Cracids, Cranes, Creepers, Crows, Cowbirds, Dickcissel, Dippers, Diving-Petrels, Dovekies, Doves, Dowitchers, Ducks, Dunlins, Eagles, Egres, Emus, Euphonias, Fairy-Wrens, Falcons, Fernwrens, Finches, Finfoots, Flamingos, Flickers, Flycatchers, Fowl, Furnarids, Frigatebirds, Frogmouths, Fulmars, Gallinules, Gannets, Geese, Goshawks, Godwits, Grackles, Grebes, Ground-Rollers, Grouse, Grosbeaks, Guam Rails, Guans, Guilemots, Guineas, Gulls, Hamerkop, Hawks, Helmet-Shrikes, Herons, Hoatzins, Honeyeaters, Hoopoes, Hornbills, Hummingbirds, Ibises, Indigobirds, Ioras, Jacamars, Jacanas, Jackdaws, Jaegers, Japanese White-Eyes, Jays, Juncos, Kago, Killdeers, Kingbirds, King Fishers, Kinglets, Kites, Kittiwake, Kiwis, Kestrels, Knots, Lapwings, Larks, Longspurs, Loons, Lories, Macaws, Magpies, Mallards, Manakins, Martins, Meadowlarks, Mejiros, Merlins, Mesites, Mimids, Moas, Mockingbirds, Motmots, Mousebirds, Murres, Murrelets, Mynahs, Night-Herons, Nutcrackers, Nuthatches, Orioles, Oropendolas, Osprey, Ostrich, Owls, Oystercatchers, Palmchats, Pardalotes, Parrots, Partridges, Peacocks & Peafowl, Pelicans, Penguins, Petrels, Phalaropes, Pheasants, Pigeons, Pittas, Plovers, Pochards, Prairie Chicken, Ptarmigans, Puffins, Quails, Quetzals, Rails, Raptors, Ravens, Razorbills, Redstarts, Rhabdornis, Rhea, Roadrunners, Robins, Rollers, Ruffs, Sanderlings, Sandgrouse, Sandpipers, Sapsuckers, Scrubfowl, Scrubwrens, Seabirds, Shorebirds, Secretarybirds, Seriemas, Shags, Shearwaters, Shrikes, Skilts, Skimmers, Snipes, Sparrows, Softbills, Spoonbills, Scrubwrens, Starlings, Stilts, Storks, Storm-Petrels, Sunbirds, Sunbittern, Sungrebes, Swallows, Swamphens, Swiftlets, Swifts, Takahe, Tanagers, Teals, Terns, Thornbills, Thrashers, Thrushes, Tinamous, Titmouses, Toucans, Towhees, Townsends, Trogons, Tropicbirds, Tucaros, Turkeys, Vangas, Veery, Verdins, Vireos, Vultures, Wagtails, Warblers, Waterthrushes, Waxwings, Weavers, Wheatears, Whimbrels, White-eyes, Willets, Woodpeckers, Woodcocks, Wrens, Wrentits, Yellowlegs, and Yellowthroats.

The siRNA methods described herein can be useful to directly to treat many types of viral diseases in humans. Examples of such treatments are shown in Examples 13-15, wherein RNAi is used to treat disease in humans ex vivo, in vivo and in utero.

The above disclosure generally describes the present invention. A more complete understanding can be obtained by reference to the following specific examples which are provided herein for purposes of illustration only and are not intended to limit the scope of the invention. A new method for inhibition of HIV replication was developed using RNA interference is described in the following examples. Additionally, a method of inhibiting RSV in chick embryos using RNAi is also described.

### EXAMPLES

In the following examples, Lipofectamine 2000 (LF2000) and Opti-MEM (OMEM) were purchased from Invitrogen (Carlsbad, Calif.). siRNAs were purchased from Dharmacom Research (Lafayette, Colo.). Deprotection and annealing of siRNA was as carried out as described by the manufacturers protocol. The lentiviral vectors used a three-plasmid expression system to generate HIV-derived retroviral vector particles by transient transfection as described for other vectors. Plasmid pCMV?R9, the packaging construct, contained the human cytomegalovirus immediate early promoter, which drove the expression of all viral proteins required in trans. This plasmid is defective for the production of the viral envelope and the accessory protein Vpu. The packaging signal and adjacent sequences were deleted from the 5′ untranslated region, but the 5′ splice donor site was preserved. A polyadenylation site from the insulin gene was substituted for the 3′ long terminal repeat at the end of the nef reading frame. This design eliminated cis-acting sequences crucial for packaging, reverse transcription, and integration of transcripts derived from the packaging plasmid.

To broaden the tropism of the vector, a second plasmid was used that encodes a heterologous envelope protein for pseudotyping the particles generated by pCMVR9. Two variants were used: one encoding the amphotropic envelope of MLV and the other encoding the G glycoprotein of VSV. The third plasmid, the transducing vector (PHR′) contained cis-acting sequences of HIV required for packaging, reverse transcription, and integration, as well as unique restriction sites for the cloning of heterologous complementary DNAs. Nearly 350 bases of gag as well as env sequences encompassing the Rev response element flanked by splice signals were included in the pHR′ vector. This increased packaging efficiency and allowed the transcription and cytoplasmic export of full-length vector transcripts only in the presence of the HIV Tat and Rev regulatory proteins, both of which were encoded by the packaging plasmid, PCMV R9.

In the absence of these transacting factors, the only detectable expression originated from the internal promoter in the vector. The firefly luciferase, green fluorescent protein, HIV gag, and HIV int were inserted into pHR′ downstream of the CMV immediate early promoter according to the methods describe by Naldini, et al., Science 272:263-267 (1996), which is incorporated herein by reference.

The following siRNA sequences were used:

siLUC-1: siRNAs Complementary to the Firefly Luciferase Gene

| | |
|---|---|
| CUUACGCUGAGUACUUCGAAA | (SEQ ID NO: 1) |
| \|\|\|\|\|\|\|\|\|\|\|\|\|\|\|\|\|\|\| | |
| GUGAAUGCGACUCAUGAAGCU | (SEQ ID NO: 2) |

siLUC-2: siRNAs Complementary to the Firefly Luciferase Gene

| | |
|---|---|
| GAGCUGUUUCUGAGGAGCCUU | (SEQ ID NO: 3) |
| \|\|\|\|\|\|\|\|\|\|\|\|\|\|\|\|\|\|\| | |
| UUCUCGACAAAGACUCCUCGG | (SEQ ID NO: 4) |

siGFP-1: siRNAs Complementary to the Green Fluorescent Protein Gene

| | |
|---|---|
| 5′-GCAAGCUGACCCUGAAGUUCAU-3′ | (SEQ ID NO: 5) |
| \|\|\|\|\|\|\|\|\|\|\|\|\|\|\|\|\|\|\|\| | |
| 3′-GCCGUUCGACUGGGACUUCAAG-5′ | (SEQ ID NO: 6) |

siGFP-2: siRNAs Complementary to the Green Fluorescent Protein Gene

| | |
|---|---|
| AGCAGCACGACUUCUUCAAGUCC | (SEQ ID NO: 7) |
| \|\|\|\|\|\|\|\|\|\|\|\|\|\|\|\|\|\|\|\|\| | |
| CUUCGUCGUGCUGAAGAAGUUCA | (SEQ ID NO: 8) |
| SiGAG (siHIV-gag): siRNA against HIV GAG: | |
| 5′-GCAUUGGGACCAGGAGCGACA-3′ | (SEQ ID NO: 9) |
| \|\|\|\|\|\|\|\|\|\|\|\|\|\|\|\|\|\|\| | |
| 3′-UUCGUAACCCUGGUCCUCGCU-5′ | (SEQ ID NO: 10) |
| siIN (siHIV-pol): siRNA against HIV INT: | |
| 5′-GGGGCAGUAGUAAUACAAGAU-3′ | (SEQ ID NO: 11) |
| \|\|\|\|\|\|\|\|\|\|\|\|\|\|\|\|\|\|\| | |
| 3′-UUCCCCGUCAUCAUUAUGUUC-5′ | (SEQ ID NO: 12) |
| siMLV-gag-1 | |
| 5′-UACUGGCCGUUCUCCUCUUTT-3′ | (SEQ ID NO. 13) |
| \|\|\|\|\|\|\|\|\|\|\|\|\|\|\|\|\|\|\| | |
| 3′-TTAUGACCGGCAAGAGGAGAA-5′ | (SEQ ID NO. 14) |
| siMLV-gag-2 | |
| 5′-CCACCUAGUCCACUAUCGCTT-3 | (SEQ ID NO. 15) |
| \|\|\|\|\|\|\|\|\|\|\|\|\|\|\|\|\|\|\| | |
| 3′-TTGGUGGAUCAGGUGAUAGCG-5′ | (SEQ ID NO. 16) |
| siRSV-gag-1 | |
| 5′-GGGUUGCUUAUGUCUCCCUCA-3′ | (SEQ ID NO. 17) |
| \|\|\|\|\|\|\|\|\|\|\|\|\|\|\|\|\|\|\| | |
| 3′-UUCCCAACGAAUACAGAGGGA-5′ | (SEQ ID NO. 18) |
| siRSV-gag-2 | |
| 5′-CGCUAAACAGUGUAGGAAGCG-3′ | (SEQ ID NO. 19) |
| \|\|\|\|\|\|\|\|\|\|\|\|\|\|\|\|\|\|\| | |
| 3′-UUGCGAUUUGUCACAUCCUUC-5′ | (SEQ ID NO. 20) |

### Example I

Identification of a Model Cell Line for Testing siRNAs

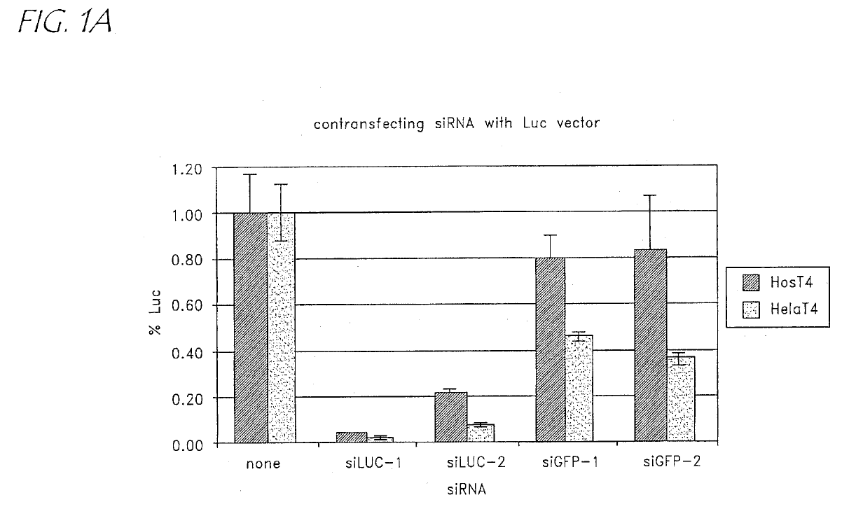

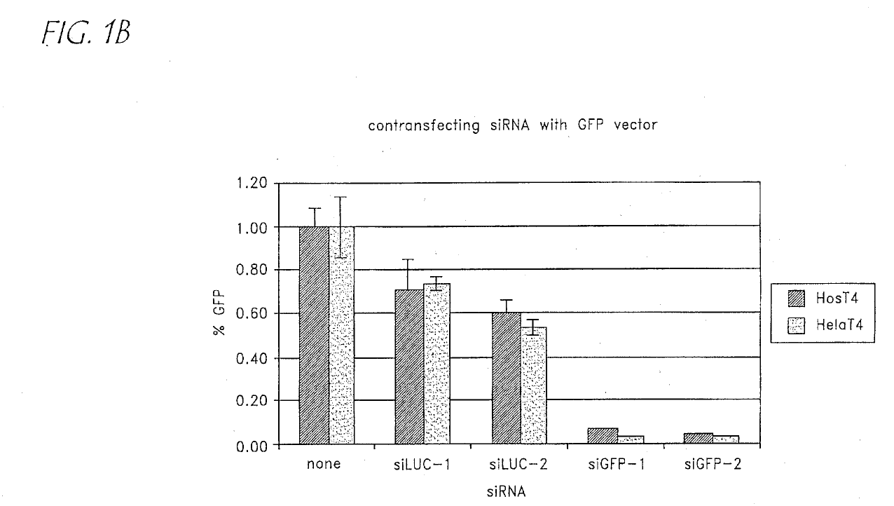

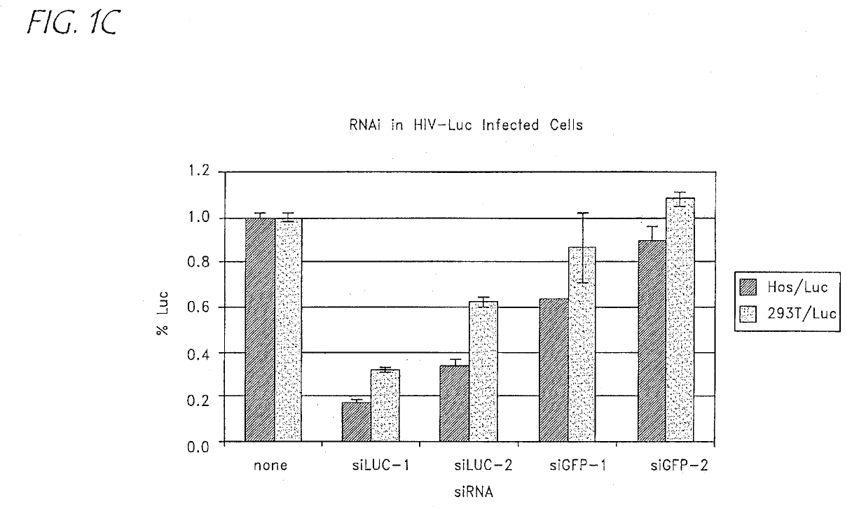

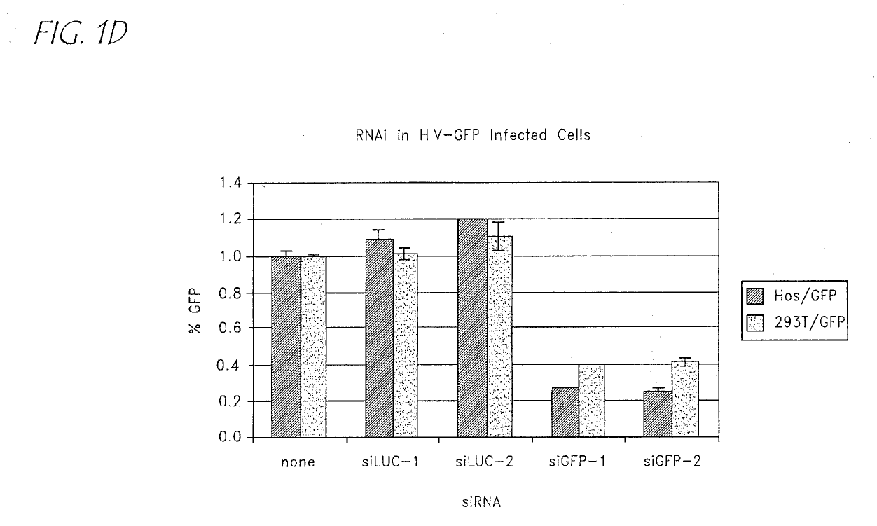

293T, HOS, HOS.T4 and Hela.T4 cells were cultured in DMEM plus 10% fetal calf serum. 5.25 cells were cultured in RPMI medium plus 10% fetal calf serum. All media also contained Penicillin, Streptomycin and Glutamine. In the experiments shown in FIGS. 1A and 1B, genes for green fluorescent protein (gfp) or fire fly luciferase (luc) were introduced into HOS.T4 cells or Hela.T4 cells by transfection (“T4” indicates that the cells are modified to express CD4, the HIV receptor). siRNAs were added to the same transfection mixtures. The siRNAs used were 21 bp in length with two-base 3′-unpaired nucleotides.

The siRNAs were transfected as follows: LF2000 transfection was carried out as described in manufacturer's protocol. 1 ml LF2000 was mixed and incubated with 50 ml OMEM for 5min before added to 50 ml OMED containing 10 nM siRNA and 0.5 mg DNA. The 100 ml mixture was incubated for 20 min at room temperature. The volume of plating medium was 0.5 ml for 12-well and 24-well plates. 5×104 to 1×105 cells were transfected for 2-4 hr in the DMEM with FBS (without antibiotics), then the medium was replaced with 1-2 ml complete medium.

The criteria for a usable cell line were satisfied by human osteosarcoma (HOS) cells, which are also advantageous since many variants have been engineered for HIV research. FIG. 1 illustrates the RNAi response of HOS and several other human cell types. Two siRNAs were tested for each gene, and both were found to be active, suppressing accumulation of the marker gene 4-20 fold. The effect was specific—the siLUC or siGFP suppressed the inappropriate marker 20-60%, in all cases substantially less than the cognate gene. This example demonstrates that RNAi is readily inducible in human 293T and HeLa cells. RNAi was also tested in the situation where the GFP or LUC were incorporated in the cellular genome using a viral vector.

### Example II

Confirmation that siRNAs can Inhibit an Integrated Heterologous Gene

The GFP or LUC genes were first incorporated into the cellular genome using a lentiviral vector such as that in Naldini et al., supra, (1996), which is incorporated herein by reference in its entirety. HOS cells and 293T cells were compared (FIGS. 1c and d). In this case, subsequent treatment of the transduced cells with the siRNAs also resulted in reduced expression of the indicator gene. The effect was quantitatively less than for the cotransfection test in FIGS. 1A and B, probably at least in part because accumulated GFP or LUC protein needed to be degraded for inhibition to be evident. Also, 293T cells have consistently shown less inhibition by RNAi than the other cells studied. This data establishes that endogenous genes can be inactivated by RNAi.

### Example III

Inhibition of HIV-1 Infection by RNAi

HIV-1 stocks were generated by transfection of 293T or HelaT4 cells using the Lipofectamine 2000 technique and following the manufacturer's protocol. After 72 hours, supernatants were collected and filtered through 0.45 micron filters. Target cells (5.25) were infected with 0.05-1 ml volume of viral supernatant in 6, 12, or 24 well plates. Infection was scored by FACS assay 48 hours after infection. Cells containing gfp or luc genes were constructed using HIV-vectors transducing the indicated gene. The self-inactivating (SIN) HIV vector (p156RRLsinPPTCMVGFPPRE) was used for GFP, the LUC transducing vector was made by substituting the GFP gene for the luc gene using standard DNA cloning methods. The SIN HIV-based vector supernatants were prepared by three-plasmid cotransfection into 293T cells with pVSVG, pdeltaR9 (Naldini et al., supra, (1996) ), and p156RRLsinPPTCMVGFPPRE (Follenzi, et al. Nature Genetics 25:217-222 (2000)). The pRRLsin.hPGK.EGFP.W pre vectors containing the cPPT sequence were generated by inserting an 118 bp HpaII-ClaI fragment obtained by PCR, using the pCMVR8.74 as template, with oligonucleotide primer:

(5′-TCGCGACCGGTTAACTTTTAAAAGAAAAGGGGGG-3′ - SEQ ID NO: 29 and
5′-AAGCTTCCGGAAAATTTTGAATTTTTGTAATTTG-3′, SEQ ID NO: 30),

followed by digestion with HpaII-ClaI. Amplification conditions were as follows: 94□C. for 4 min, then 30 cycles of 94□C. for 1 min, 54?C. for 1 min and 72□C. for 1 min, followed by extension at 72□C. for 10 min. Titers of the HIV-GFP vector were measured as the number of infectious units forming GFP-positive centers on 293T cells per ml. Typical vector stocks were about 3×107 infectious units per ml. For a typical infection, 5-100 ml HIV-cPPT-GFP (P24: 78 ng/ml) or 1 ml HIV-cPPT-LUC (P24: 241 ng/ml) were used to infect 293T cells (1×104 cells per 100 ml well in a 96 well plate.

siRNAs were designed against sequences in HIV gag (siGAG), (SEQ ID Nos 9 and 10) or HIV integrase (siIN) (SEQ ID Nos 11 and 12). RNA molecules were 21 bases in length. siRNAs were transfected into HOS cells together with plasmids encoding either of two HIV strains, R9 or NL4-3. Virus-containing culture supernatant was collected after 2 days and used to infect 5.25 indicator cells. These cells contain a luc gene under control of an HIV LTR, so luc transcription is induced only when cells are supplied with HIV-1 Tat from an integrated provirus. Thus, luciferase activity is a quantitative marker for infection. As a control, a nonspecific siRNA (siGFP-1, SEQ ID NOs: 5 and 6) was also compared with siGAG (SEQ ID NOs: 9 and 10) and siIN (SEQ ID NOs: 11 and 12) for inhibition of HIV-1.

As can be seen in FIG. 4, HIV production was inhibited >95% by siGAG (SEQ ID NOs: 9 and 10) or siIN (SEQ ID NOs: 11 and 12) but not sGFP-1. The effect is specific, since the siGFP control did not inhibit HIV production. In FIG. 4, “Control” indicates no HIV-encoding DNA added to the transfection. The siGAG (SEQ ID NOs: 9 and 10) is complementary to the GAG gene of HIV. The siIN (SEQ ID NOs: 11 and 12) is complementary to the IN (integrase) gene of HIV, and siGFP-1 (SEQ ID NOs: 5 and 6) is complementary to the green fluorescent protein gene of the jellyfish Aequoria.

These data establish that siRNA directed against HIV can effectively block production of HIV virus. The effect of siRNA also was monitored by assaying accumulation of p24 capsid antigen in the culture medium of the transfected HOS cells.

### Example IV

siRNA Half-Life

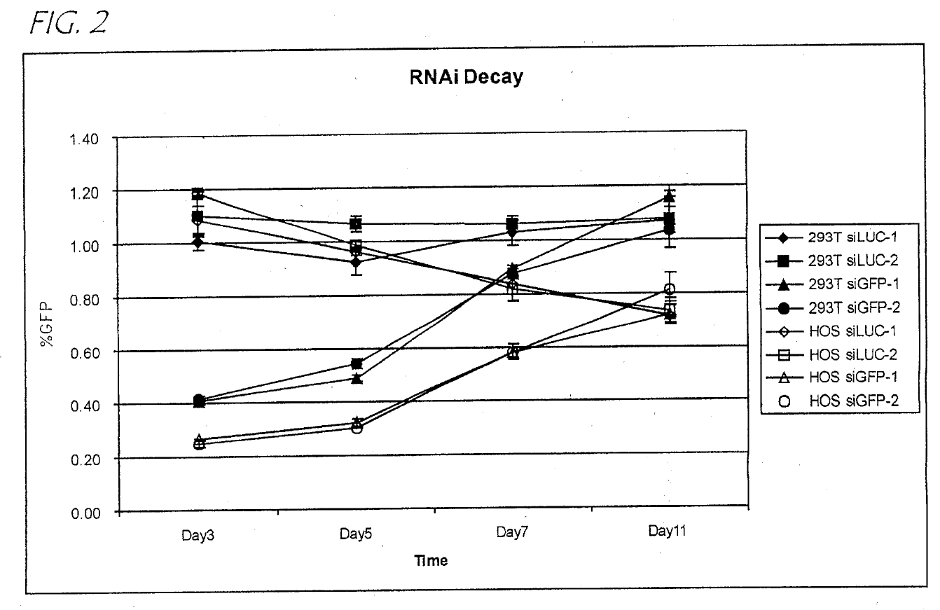

To examine the half life of the inhibitory effect, RNAi was induced against GFP with siGFP-1 in cells containing an integrated HIV-GFP vector and inhibition was assayed as a function of time after treatment (FIG. 2). The observed half-life of the inhibitory effect was about 7 days. Thus, although RNAi-mediated inhibition did not persist indefinitely in the cell culture system assayed, the lifetime compared favorably with many small molecules used therapeutically.

In summary, RNAi against HIV can be induced in HOS cells, blocking HIV production by more that 95%. The inhibition was only seen when the siRNA matched sequences in the HIV RNA—a nonspecific siRNA was not inhibitory. These findings establish that RNAi can program the selective destruction of a pathogen in human cells. Thus the RNAi system qualifies as a new branch of the human adaptive immune system, programmed by RNA instead of by antigen. Since all human cell lines tested to date are inducible for RNAi, RNAi can be adapted for therapy of infectious disease in humans.

### Example V

RNAi blocks Retroviral Gene Expression Late during Infection

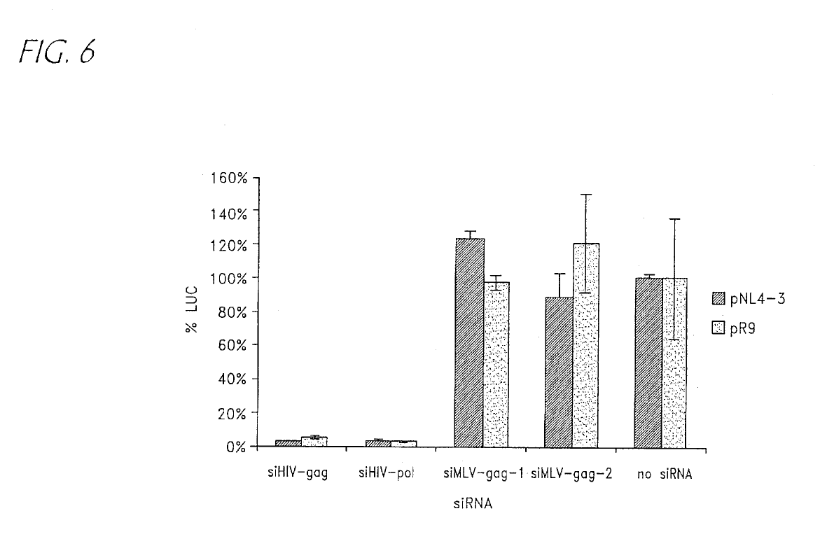

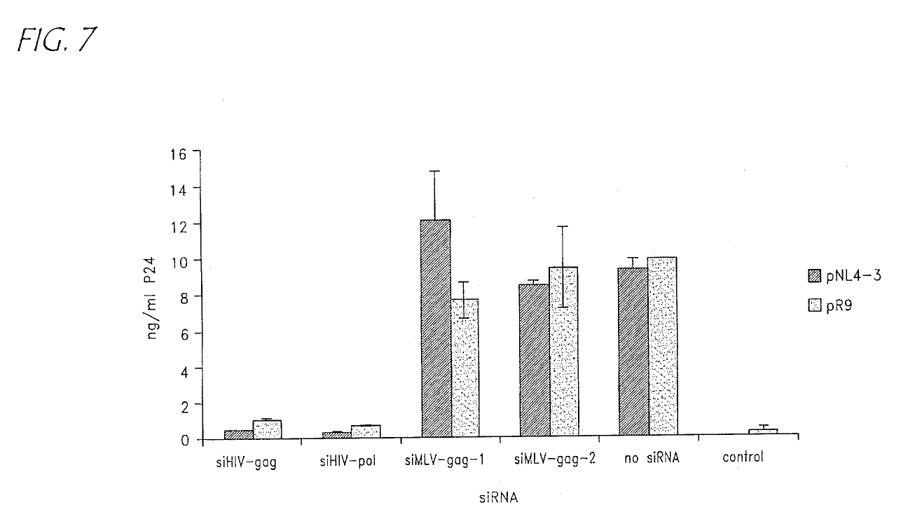

This example demonstrates the use of the RNAi system to determine which step in HIV replication is affected by the siRNAs. Inhibition of late viral transcription can be assayed by cotransfecting siRNAs with a cloned HIV 1 provirus. Introducing the HIV genome in this way bypasses the early steps of entry, reverse transcription, and integration and allows the effects on the late steps to be analyzed in isolation. The effects on two different HIV-1-encoding plasmids were compared (pR9 and pNL4-3). Viral particles produced from transfected HOS. T4.XCR4 cells were analyzed by infecting 5.25luc4 indicator cells (FIG. 6) or by quantifying p24 capsid production (FIG. 7). siRNAs against HIV-1 were highly inhibitory in this setting (>95%), while control siRNAs (siMLV-gag-1 and siMLV-gag-2 (both shown in FIG. 6) and siGFP (not shown)) did not inhibit virus production. Further, Northern blot analysis revealed that siHIV-1 and siHIV-2 specifically reduced accumulation of the viral genomic RNA.

### Example VI ps RNAi does not Act Early Against Incoming Viral RNA Genomes

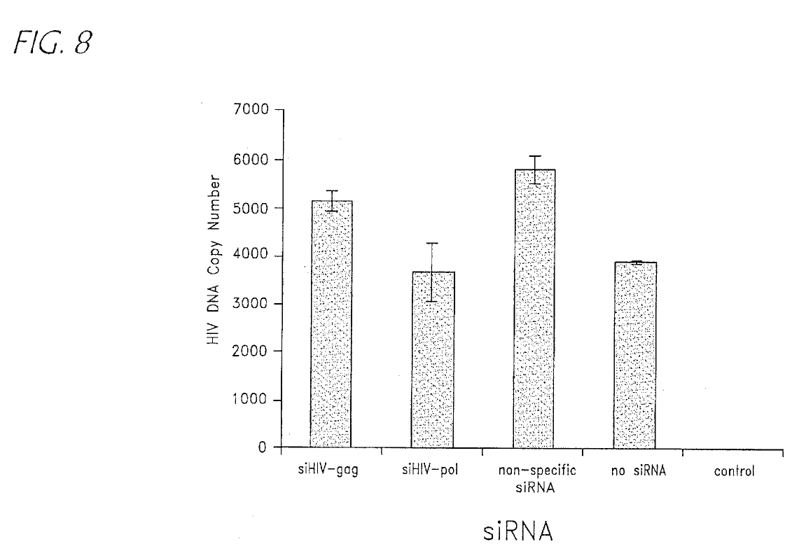

To assay the effect of siRNA on the early steps of infection, HOS.T4.CXCR4 cells were infected with HIV-1 and the accumulation of reverse transcription products was quantified. Efficient reverse transcription requires an intact RNA template, so possible action of RNAi should result in reduced accumulation of viral cDNA. DNA samples were harvested from cells 12 hr after infection, a time at which reverse transcription products are known to peak in abundance. Viral cDNA copies were quantified by fluorescence-monitored quantitative PCR with primers that require the two template transfers of reverse transcription to be completed for the amplicon to be produced (Butler et al., (2001) Nat. Med. 7:631-634). No significant differences were detected between siRNA-treated and nontreated cells, and this reveals that viral RNA genomes packaged in early replication complexes were not efficient substrates for RNAi (FIG. 8). Thus, RNAi was active against mRNAs transcribed late during infection from proviral DNA but did not attack RNA genomes early after entry.

The following examples describe RNAi in Chick Embryos. The chick embryo has served as a classical system for studying the cellular interactions that control development. Though many tools are in place for studying this system, what has been lacking is the ability to inhibit gene expression. The following examples provide evidence that chick embryos, in particular the cells in the developing neural tube that give rise to the spinal cord, contain the cellular machinery to use siRNAs as a substrate for the targeted degradation of mRNAs.

### Example VII

Tissue Electroporation of Chick Embryos

Chick embryos were utilized as a model system to evaluate the activity of RNAi against avian retroviruses. In initial studies, a plasmid encoding GFP was introduced into the chick neural tube, together with siRNA against gfp (siGFP). As a control, siRNAs against irrelevant sequences were also tested to document the specificity. Short 21- to 23-bp double-stranded siRNAs were designed as described in Elbashir et al., (2001) EMBO J. 20:6877-6888, and were synthesized chemically.

Tissue electroporation efficiently introduced the nucleic acids into the half of the neural tube proximal to the positive electrode and occasionally into neural crest cells that migrated from the neural tube and differentiated into structures such as sensory neurons of the dorsal root ganglion (DRG) (Inoue and Krumlauf (2001) Nat. Neurosci. 4:1156-1157). The Chick embryos (SPAFAS, Mcintyre Farms) were incubated in a humidified chamber and were staged according to Hamburger and Hamilton (H.H.). H.H. stage 11 chick embryos were windowed, DNA (0.5-3.0 μg/μl) and siRNA (0.1-1.1 μg/μl) were pipetted into the lumen of the neural tube, and the electrodes were placed on either side of the neural tube over the vitelline membrane. A square wave electroporator (BTX) was used to administer five pulses of current at 25V for 50 ms each (Nakamura et al., (2001) Methods 24:43-48). Eggs were sealed, and the embryos were allowed to develop to H.H. stage 23.

### Example VIII

Demonstration of RNAi Activity in Chick Embryos

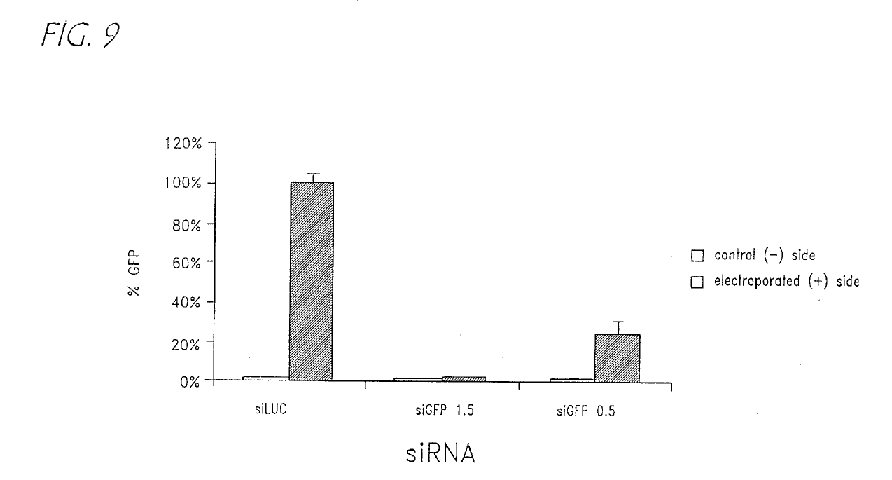

Two days after electroporation, the embryos were then prepared for immunocytochemical analysis. Whole-mount GFP-transfected chick embryos were photographed with a Zeiss Stemi SV fluorescent dissecting microscope. Extensive fluorescence was seen in the neural tube of the control siLUC-treated and in embryos electroporated with the gfp plasmid only. Treatment with 1.5 μg/μl siGFP, in contrast, greatly abrogated the fluorescent signal. Treatment with lower doses of siGFP (0.5 μg/μl) resulted in weaker inhibition. Treatment with either RNA strand alone did not inhibit the appearance of the fluorescent signal, indicating that the RNAs were not working by an antisense mechanism. Embryos were then sectioned through the spinal cord, and GFP expression was assayed by fluorescence microscopy. The intensity of GFP fluorescence is quantitated in FIG. 9. An intense GFP signal is seen in the electroporated half of the embryo, and the signal is reduced in a concentration-dependent fashion by coelectroporation of siGFP.

To visualize the tissue organization and assess the specificity of RNAi, embryos were also stained with an antibody recognizing the endogenous nuclear proteins IsI1 and IsI2 expressed by motor neurons and DAG. No increase in embryo mortality was observed due to the addition of RNAi up to a concentration of 3 5 μg/μl, and motor neuron differentiation proceeded normally, as monitored by antibody staining for IsI1/2. These data indicate that the chick neural tube contains the machinery for RNAi and that RNAi can be elicited without toxicity by tissue electroporation of siRNAs.

### Example IX

Control of RSV Infection by RNAi

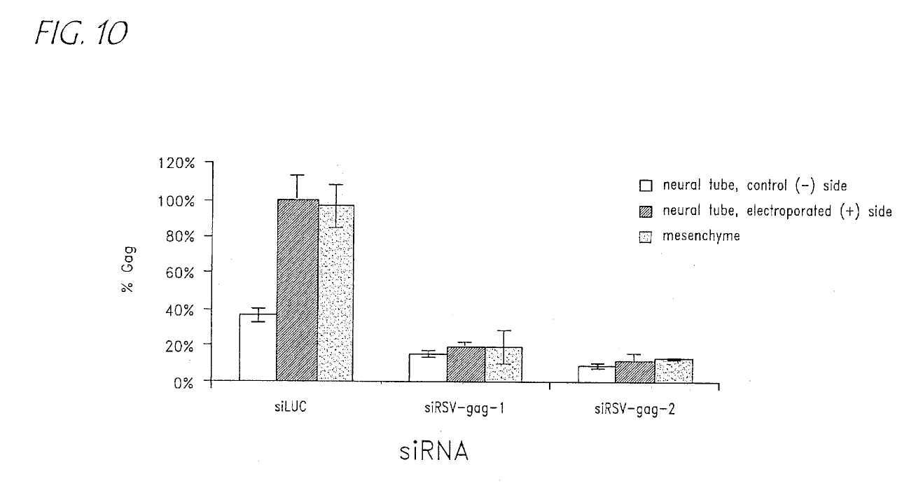

To determine whether RNAi could inhibit retroviral replication, the RCASBP(B) virus was used as a model. RCASBP(B) is a derivative of RSV modified for use as a retroviral vector by removal of the src oncogene (Hughes, et al., (1987) J. Virol. 61:3004-3012). RCASBP(B) is known to be competent for replication in the chick neural tube. Embryos were electroporated with a plasmid encoding the RCASBP(B) genome and either of two siRNAs against RSV Gag (siRSV-gag-1 and siRSV-gag-2) or siLUC-1 as a nonspecific siRNA control. Electroporation was carried out in embryos 2 days after fertilization and was analyzed at day 4 by sectioning and staining with an antibody against RSV Gag. As shown in FIG. 10, after treatment with the RCASBP(B) plasmid only (not shown), or with RCASBP(B) and the siLUC-1 control RNA, Gag staining was observed in one half of the spinal cord. The lumen of the neural tube and the unilateral electroporation method restricted the infection to one half of the cord; however, the replication-competent virus did spread into the surrounding mesenchyme due to secondary cell infection. After electroporation of the RSV genome with either of the siRSVs, infection was only evident in a few cells, indicating efficient inhibition of viral replication. Much less viral spread was evident at the 2 day postelectroporation time point. There was little virus evident in the nonelectroporated half of the neural tube, apparently because the lumen of the tube and surrounding membranes formed a barrier to viral spread. Quantitation of the intensity of the Gag signal (FIG. 10) suggested that siRSV-gag-2 is a somewhat more effective inhibitor than siRSV-gag-1. Electroporation of single strands of the siRNAs did not inhibit viral replication. These findings indicate that RNAi can suppress RSV replication efficiently in chick embryos.

### Example X

Inhibition of RSV Pathogenesis by RNAi

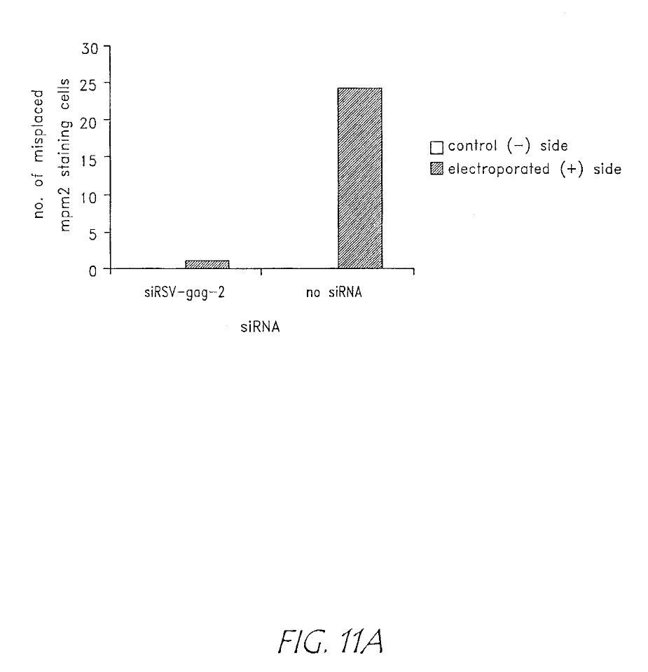

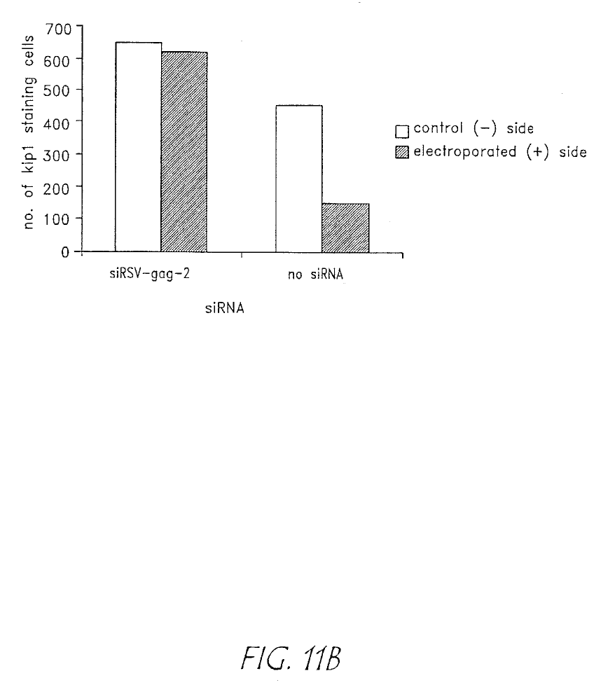

To find out whether RNAi could inhibit pathogenesis by RSV in the chick embryo model, the following experiment was performed. RSV was electroporated into 2-day-old embryos, and the effects were assessed after another 3 days of incubation. All embryos electroporated with RSV only or RSV plus control siRNA were dead by this time (16/16 and 12/12 assayed, respectively). In contrast, 7/12 embryos treated with siRSV-gag-2 survived, indicating inhibition of the lethal effect by RNAi. To assay pathogenesis in more detail, embryos were sectioned 36 hr after infection and were stained with markers for mitotic cells (mpm2), non proliferative cells (kipi), and RSV Gag (FIG. 11). In the absence of specific siRNA, the neural tube was disorganized, with misplaced proliferative cells evident. In the presence of siRSV-gag-2, the embryos were indistinguishable from embryos that were not infected with RSV. These data confirm that RSV causes abnormal proliferation and tissue disorganization in embryos, and this effect can be reversed with RNAi.

FIG. 11A is a bar graph showing the quantitation of abnormal cells stained with the mpm2 marker, comparing staining on the electroporated (+) and control (−) sides of the embryo. The mpm2 marker detects tyrosine phosphorylation characteristic of mitosis. Cells outside the normal axial zone of proliferation were summed over four slides. The mpm2 marker detects cells in a specific phase of the proliferative cycle and so stains relatively low numbers in any given section. FIG. 11B is a bar graph showing the quantitation of cells stained with the kip1 marker, comparing staining on the electroporated (+) and control (−) sides of the neural tube. The kip1 marker detects the kip1 protein that inhibits cell cycle progression and so marks postmitotic cells. Cells were counted in four sections for each bar graph.

### Example XI

RNAi Inhibits the Late Steps of RSV Replication

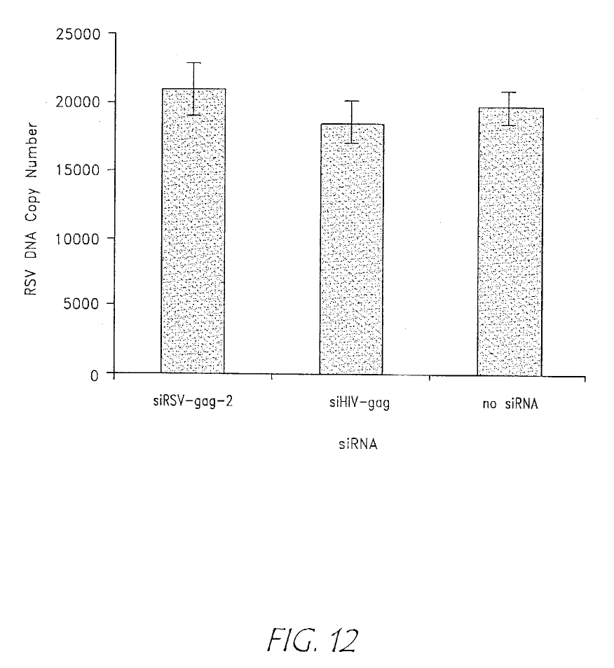

The inhibition of the late steps of RSV replication by RNAi was tested (FIG. 12). Chicken DF-1 cells were transfected with siRSV-gag-1, siRSV-gag-2, or nonspecific siRNAs, and then infection was initiated by transfection with a plasmid encoding RCASBP(B). Two days later, RSV particles were harvested from cell supernatants and were analyzed by Western blot for accumulation of RSV capsid proteins. siRSV-gag-1 and siRSV-gag-2 showed inhibition in both settings. The single strands of siRSV-1 or siRSV-2 were not inhibitory. Inhibition of RSV replication by RNAi was also found in experiments in which RSV genomes were introduced by infection rather than transfection.

To assess the effects of siRSV on the early steps of infection, DF-1 cells were treated with siRSV-gag-2 or siHIV-gag as a control and were infected with RCASBP(B), and reverse transcription was measured 12 hr later (FIG. 12). The number viral genomes was quantitated by using real-time PCR (Taqman). As shown for HIV in FIG. 8, there was no significant inhibition of cDNA accumulation by specific siRNAs. These data indicate that RNAi acts primarily on RSV messages produced late during infection and, together with data on HIV, suggests that this can be generally true of retroviruses.

### Example XII

Quantitative PCR Assays of Viral cDNA Synthesis

The total DNA was purified at 12 hr after infection with the QIAGEN DNeasy kit and was suspended in 25 ng/μl final concentration. Quantitative PCR was carried out as described in Butler et al (2001) Nat. Med. 7:631-634.

The primers and probes used for quantitative PCR are listed below:

| | |
|---|---|
| Primer | Sequence (5′ to 3′) |

| | | |
|---|---|---|
| RSV-src-SalI | GAGAGCGTCGACAGCACACAAGGTAGTT | (SEQ ID NO. 21) |
| RSV-src-ClaI | CCATCGATGAAGCAGCGCAAAACGCCTAAC | (SEQ ID NO. 22) |
| HIV-F | TGTGTGCCCGTCTGTTGTGT | (SEQ ID NO. 23) |
| HIV-R | GAGTCCTGCGTCGAGAGAGC | (SEQ ID NO. 24) |
| HIV-probe | (FAM)-CAGTGGCGCCCGAACAGGGA-(TAMRA) | (SEQ ID NO. 25) |
| RSV-F | CCCCGACGTGATAGTTAGGG | (SEQ ID NO. 26) |
| RSV-R | CGAGACGGATGGAGACAGGA | (SEQ ID NO. 27) |
| RSV-probe | (FAM)-TCGGCCACAGACGGCGTGG-(TAMNph) | (SEQ ID NO. 28) |

Of particular importance is the use of siRNA molecules to treat viral diseases in humans. In Examples 13-15, the RNAi method is used to treat disease in humans ex vivo, in vivo and in utero.

### Example XIII

Treatment of Chickens with RNAi to Prevent RSV Infection

A synthetic hairpin dsRNA oligonucleotide is prepared using synthetic altered oligonucleotides that have increased resistance to degradation. These dsRNA oligonucleotides are mixed with feed material and fed to flocks of chickens. The chickens are later tested for their ability to prevent RSV infection at various developmental stages. Offspring of the dsRNA-treated chickens can also be tested.

### Example XIV

Treatment of RSV-infected Chicken with RNAi to Decrease Viral Pathogenicity

An RNAi-inducing sequence is engineered into a viral vector capable of self replicating in chicken tissues. When expressed in the cell, the sequence is capable of folding to form a hairpin structure. The viral vector containing the sequence of interest is mixed with feed material. Alternatively, liposome technology, biolistics, or even infection with an abrasion-mediated technique can be useful to allow entry of the vector into the tissue. Any method can be utilized such that the viral vector can enter a cell of the infected chicken. The RSV infected chickens are examined daily for indications of RSV infections, and are additionally tested for expression of RSV-related proteins. This method can be altered to treat any bird having a viral infection, and indeed, can be used to treat other types of animals such as pigs, goats, cattle, and humans.

### Example XV

Production of Transgenic Chickens Capable of Constitutive Expression dsRNA Molecules to Prevent RSV Pathogenicity

A nucleic acid encoding a dsRNA hairpin is engineered into a heritable but replication-defective viral vector capable of chromosomal integration. The vector is micro-injected under the surface of unincubated chicken embryo blastoderms, following, for example, the method of Briskin et al. (1991) Proc. Natl. Acad. Sci. USA. 88:1736-1740, which is hereby incorporated by reference in its entirety. The embryos are later tested to see which ones have stably integrated the hairpin encoding sequence into their chromosomes, and are further tested to ensure expression of the hairpin sequences. The chickens are tested for their ability to prevent RSV pathogenicity. These transgene-carrying chickens are allowed to breed, and the offspring tested for those that carry the transgene on both sets of chromosomes. These offspring are used as founder birds to breed larger flocks of chickens, each of which carries two sets of RNAi-inducing nucleic acid sequences. The future offspring of these founder birds should be protected from RSV pathogenesis.

### Example XVI

Treatment of Pigeons to Prevent West Nile Virus

Genes essential for replication of west nile virus are identified and chosen. An RNA corresponding to a segment of the chosen target gene is prepared such that it folds over to form a dsRNA “hairpin”. Alternatively, the sequence can be prepared using synthetic oligonucleotides that allow for increased stability of the dsRNA sequence. The sequence is mixed with pigeon feed material at varying concentrations. The pigeons are then challenged with west nile virus, and later tested for the ability to prevent viral pathogenicity.

### Example XVII

Production of Transgenic Pigs having RNAi-mediated Inhibition of Parvovirus Pathogenicity

A target parvovirus gene is identified which is essential for parvoviral replication. A DNA sequence is designed such that, when linked to a suitable promoter and integrated into the swine chromosome, the RNA is transcribed and is able to form a double-stranded hairpin structure, which corresponds to a portion of the target parvovirus gene.

The engineered nucleic acid sequence is inserted to a plasmid vector that allows stable integration into the cellular chromosomes-of pigs. The nucleic acid is microinjected into the nucleus of pig ova following the method of Wall et al., 1991 (Proc. Natl. Acad. Sci. USA. 88:1696-1700), which is hereby incorporated by reference in its entirety. Transgenic piglets expression the dsRNA sequence are identified using southern blotting of DNA prepared from tail biopsies. Transgenic piglets are crossed, and the F1 generation is analyzed using PCR primers specific for the dsRNA fragment. The transgenic pigs are used as founders for future breeding. The transgenic pigs are challenged with parvovirus. The test pigs are later tested for parvovirus pathogenicity and symptomology.

### Example XVIII

Treatment of HIV with RNAi

10⁸ bone marrow cells are removed from a patient with HIV and cultured in vitro. A lentiviral vector engineered to encode a stem-loop RNA complementary to siGAG (SEQ ID NOs: 9 and 10) is transfected into the bone marrow cells. The infected cells are then reintroduced into the patient. The HIV load is monitored using standard ELISA and PCR tests. The patient's symptoms are monitored. After the patient has recovered from this procedure (in about 1 month), a second treatment is conducted. In the second treatment, siRNAs corresponding to siIN (SEQ ID NOs: 11 and 12) are introduced by “GeneGun”. The siIN siRNAs are initially affixed to beads and the beads ballistically introduced into muscle using the Gene Gun. This procedure is repeated every 3 days to 1 week until 6 months after absolute clearance of the virus by PCR.

### Example XIX

Treatment of Tuberculosis

SiRNAs complementary to the mycobacterial RNA polymerase are packaged into liposomes and inhaled into the lungs of a patient with tuberculosis. The process is repeated at 3 -7 day intervals until the bacteria is cleared from the body. The process is effected in conjunction with antibiotic therapy, such as rifampin.

### Example XX

Treatment of HIV In Utero

siRNAs corresponding to siIN (SEQ ID NOs: 11 and 12) and or siGAG (SEQ ID NOs: 9 and 10) are packaged into vesicles and introduced by injection into the uterus at 3 to 7 day intervals until birth. At this time the baby is tested for viral load and RNAi therapy is continued, if necessary.

Throughout this application various publications have been referenced. The disclosures of each of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

We claim:

1. A method for inhibiting the growth of a pathogen comprising contacting the pathogen with a double-stranded RNA (dsRNA) molecule that corresponds to a target gene essential to growth of the pathogen; and incubating said dsRNA molecule and said pathogen under conditions suitable for RNA interference, thereby inhibiting the growth of said pathogen.

2. The method of claim 1, wherein said pathogen is contained in a cell.

3. The method of claim 2, wherein said pathogen is contacted in vivo.

4. The method of claim 1, wherein said pathogen is a virus.

5. The method of claim 4, wherein said virus is a retrovirus.

6. The method of claim 5, which said retrovirus is HIV.

7. The method of claim 5, wherein said virus is selected from the group consisting of ALV-J (avian leukosis virus, subtype J) and Rous Sarcoma Virus (RSV).

8. The method of claim 3, wherein said pathogen causes a disease upon infecting an organism.

9. The method of claim 8, wherein said organism is a vertebrate.

10. The method of claim 9, wherein said organism is a mammal.

11. The method of claim 9, wherein said organism is a bird.

12. The method of claim 9, wherein said organism is a chicken.

13. The method of claim 2, wherein said target gene is a cellular gene.

14. The method of claim 4, wherein said target gene is a viral gene.

15. The method of claim 6, wherein said target gene is an HIV gene.

16. The method of claim 15, wherein said HIV gene is gag, pol or env.

17. The method of claim 2, wherein said contacting is by a method selected from the group consisting of microinjection, transfection, viral infection, electroporation, and gene gun particle bombardment.

18. The method of claim 1, wherein said dsRNA is encoded by a viral vector.

19. A composition comprising dsRNA that corresponds to a target gene of the HIV genome.

20. The composition of claim 19, wherein said target gene is selected from the group consisting of gag, pol and env.

21. The composition of claim 24, wherein said target gene is gag.

22. The composition of claim 21, wherein said dsRNA comprises a sequence that is a combination of SEQ ID NO: 9 and 10.

23. The composition of claim 22, wherein said target gene is pol.

24. The composition of claim 23, wherein said target gene encodes integrase (IN).

25. The composition of claim 24, wherein said dsRNA comprises a sequence that is a combination of SEQ ID NOS: 11 and 12.

26. A method for identifying a gene sequence that is a target for RNA interference aimed at inhibiting the growth of a pathogen, said method comprising the steps of:
(a) selecting a candidate target gene sequence;
(b) contacting a host cell containing a pathogen with a dsRNA that corresponds to the target gene sequence; and
(c) determining whether the dsRNA inhibits the growth of said pathogen.

27. The method of claim 26, wherein said pathogen is a virus.

28. The method of claim 27, wherein said virus that causes a disease in vertebrates.

29. The method of claim 28, wherein said virus causes a disease in mammals.

30. The method of claim 28, wherein said virus that causes a disease in birds.

31. The method of claim 26, wherein said target gene sequence is cellular.

32. The method of claim 27, wherein said target gene sequence is viral.

33. The method of claim 26, wherein said contacting said contacting occurs by a method selected from the group consisting of microinjection, transfection, viral infection, electroperation, and gene gun particle bombardment.

34. The method of claim 26, wherein said dsRNA is contained on a viral vector.

35. A method for inhibiting the growth of a pathogen in an organism, comprising administering to the organism a double-stranded RNA (dsRNA) molecule that corresponds to a target gene, wherein said target gene is essential to growth of the pathogen.

36. The method of claim 35, wherein said organism is a vertebrate.

37. The method of claim 36, wherein said vertebrate is selected from the group consisting of mammals, birds, amphibians, reptiles, and fish.

38. The method of claim 37, wherein said mammal is selected from the group consisting of dogs, cats, pigs, cows, sheep, goats, guinea pig, rabbits, rats, mice, chimpanzees and humans.

39. The method of claim 38, wherein said vertebrate is a bird.

40. The method of claim 39, wherein said bird is a chicken or a turkey.

41. A method of treating a pathogenic condition in a host organism, said method comprising the steps of:
(a) identifying the pathogen causing the condition;
(b) determining a suitable target gene sequence for RNA interference that is aimed at inhibiting the growth of the pathogen; and
(c) contacting said organism with a dsRNA sequence that corresponds to said target gene sequence under conditions suitable for RNA interference, thereby treating the pathogenic condition.

42. The method of claim 41, wherein said target gene corresponds to a host cellular gene.

43. The method of claim 41, wherein said target gene corresponds to a pathogen gene.

44. The method of claim 41, wherein said pathogen is a virus.

45. The method of claim 41, wherein said contacting is effected with a viral vector.

46. The method of claim 41, wherein said host organism is a vertebrate.

47. The method of claim 46, wherein said vertebrate is a mammal.

48. The method of claim 46, wherein said vertebrate is a bird.

49. The method of claim 48, wherein said bird is a chicken.

50. The method of claim 47, wherein said mammal is selected from the group consisting of dogs, cats, pigs, cows, sheep, goats, guinea pig, rabbits, rats, mice, chimpanzees and humans.

51. A method of making a transgenic organism capable of expressing a dsRNA that corresponds to a target gene in a pathogen, said method comprising the steps of:
identifying a target gene in said pathogen;
preparing a nucleic acid sequence having a region that corresponds to a portion of the target gene, wherein the nucleic acid is able to form a double-stranded transcript once expressed in the organism;
contacting a recipient organism with said nucleic acid;
producing one or more offspring of said recipient organism;
and testing the offspring for expression of said double-stranded transcript.

52. The method of claim 51, wherein said nucleic acid is contained on a vector.

53. The method of claim 51, wherein said recipient organism is a pre-implantation mammalian embryo.

54. The method of claim 53, wherein said transformed pre-implantation embryo is transferred into a pseudo-pregnant female.

55. The method of claim 54, further comprising the step of allowing said embryo to develop into at least one viable transgenic mammal in which the expression of said target gene is inhibited by the presence of said double-stranded target gene transcript.

56. A transgenic mammal produced by the method of claim 55.

57. The method of claim 51, wherein said organism is a vertebrate.

58. The method of claim 57, wherein said organism is a bird.

59. The method of claim 58, wherein said bird is a chicken.

60. The method of claim 58 or 59, wherein said animal is contacted with primordial germ cells transfected with said nucleic acid.

61. The method of claim 60, wherein said contacting is effected by microinjection.

62. The method of claim 61, wherein said nucleic acid sequence is expressed of an inducible promoter.

63. A transgenic bird produced by the method of claim 62.

64. A transgenic chicken produced by the method of claim 62.


SEQUENCE LISTING (20 sequences)

SEQ ID NO: 1 - RNA, 21 nt, Lampyridae
cuuacgcugaguacuucgaaa

SEQ ID NO: 2 - RNA, 21 nt, Lampyridae
gugaaugcgacucaugaagcu

SEQ ID NO: 3 - RNA, 21 nt, Lampyridae
gagcuguuucugaggagccuu

SEQ ID NO: 4 - RNA, 21 nt, Lampyridae
uucucgacaaagacuccucgg

SEQ ID NO: 5 - RNA, 22 nt, Aequorea victoria
gcaagcugacccugaaguucau

SEQ ID NO: 6 - RNA, 22 nt, Aequorea victoria
gccguucgacugggacuucaag

SEQ ID NO: 7 - RNA, 23 nt, Aequorea victoria
agcagcacgacuucuucaagucc

SEQ ID NO: 8 - RNA, 23 nt, Aequorea victoria
cuucgucgugcugaagaaguuca

SEQ ID NO: 9 - RNA, 21 nt, Homo sapiens
gcauugggaccaggagcgaca

SEQ ID NO: 10 - RNA, 21 nt, Homo sapiens
uucguaacccugguccucgcu

SEQ ID NO: 11 - RNA, 21 nt, Homo sapiens
ggggcaguaguaauacaagau

SEQ ID NO: 12 - RNA, 21 nt, Homo sapiens
uuccccgucaucauuauguuc

SEQ ID NO: 13 - DNA, 21 bp, Moloney Murine Leukemia Virus
uacuggccguucuccucuutt

SEQ ID NO: 14 - DNA, 21 bp, Moloney Murine Leukemia Virus
ttaugaccggcaagaggagaa

SEQ ID NO: 15 - DNA, 21 bp, Moloney Murine Leukemia Virus
ccaccuaguccacuaucgctt

SEQ ID NO: 16 - DNA, 21 bp, Moloney Murine Leukemia Virus
ttgguggaucaggugauagcg

SEQ ID NO: 17 - RNA, 21 nt, Rous Sarcoma Virus
ggguugcuuaugucucccuca

SEQ ID NO: 18 - RNA, 21 nt, Moloney Murine Leukemia Virus
uucccaacgaauacagaggga

SEQ ID NO: 19 - RNA, 21 nt, Moloney Murine Leukemia Virus
cgcuaaacaguguaggaagcg

SEQ ID NO: 20 - RNA, 21 nt, Moloney Murine Leukemia Virus
uugcgauuugucacauccuuc